SEQUENCE LISTING (25 sequences)

SEQ ID NO: 1 - DNA, 21 bp, Mycobacterium tuberculosis
cttgagcggggtgcactcatc

SEQ ID NO: 2 - DNA, 21 bp, Mycobacterium tuberculosis
gttgagtgcatcaggctcagc

SEQ ID NO: 3 - DNA, 21 bp, Mycobacterium tuberculosis
  misc_feature (8)..(14): any “n” = any nucleotide
cttgagtnnnnnnnactcaag

SEQ ID NO: 4 - DNA, 28 bp, Artificial Sequence
  Synthetic primer
cgggatccctgttcagtcagcacaccct

SEQ ID NO: 5 - DNA, 28 bp, Artificial Sequence
  Synthetic primer
gctctagatgtggccgacgagactccca

SEQ ID NO: 6 - DNA, 28 bp, Artificial Sequence
  Synthetic primer
gaagatctatgaacgcgcacctgctgca

SEQ ID NO: 7 - DNA, 28 bp, Artificial Sequence
  Synthetic primer
gaagatctatatccacaatccgctcggt

SEQ ID NO: 8 - DNA, 40 bp, Artificial Sequence
  Synthetic nucleotide
gctcagtaagttgagtgcatcaggctcagctctgaattga

SEQ ID NO: 9 - DNA, 40 bp, Artificial Sequence
  Synthetic nucleotide
gtcaattcagagctgagcctgatgcactcaacttactgag

SEQ ID NO: 10 - DNA, 27 bp, Artificial Sequence
  Synthetic primer
ggactagtcgttgtggacgcggaggtg

SEQ ID NO: 11 - DNA, 28 bp, Artificial Sequence
  Synthetic primer
gctctagaccccgtcctttgggttcttc

SEQ ID NO: 12 - DNA, 27 bp, Artificial Sequence
  Synthetic primer
ggactagtcaccgccctggtcgtctgg

SEQ ID NO: 13 - DNA, 27 bp, Artificial Sequence
  Synthetic primer
gctctagatcagtggcaccgtcttggc

SEQ ID NO: 14 - DNA, 18 bp, Artificial Sequence
  Synthetic primer
cctcctgaatatgtagag

SEQ ID NO: 15 - DNA, 21 bp, Artificial Sequence
  Synthetic primer
tcggtcaagctggcggactga

SEQ ID NO: 16 - DNA, 20 bp, Artificial Sequence
  Synthetic primer
agccatggtgaatcctcctg

SEQ ID NO: 17 - DNA, 27 bp, Bacillus subtilis
  misc_feature (10)..(18): any “n” = any nucleotide
ttagcactcnnnnnnnnngagtgctaa

SEQ ID NO: 18 - DNA, 37 bp, Mycobacterium sp.
aggtggaacttaagcgtggtcgactcaggttcttggt

SEQ ID NO: 19 - DNA, 37 bp, Mycobacterium sp.
ctcagtaagttgagtgcatcaggctcagctctgaatt

SEQ ID NO: 20 - DNA, 37 bp, Mycobacterium sp.
gaggcaagcttgagcggggtgcactcatcatagtgca

SEQ ID NO: 21 - DNA, 37 bp, Mycobacterium sp.
tgggtaaaattgagcggaacagactcaacattgacgg

SEQ ID NO: 22 - DNA, 39 bp, Mycobacterium sp.
gaataacgttggcactcgcgaccggtgagtgctaggtcg

SEQ ID NO: 23 - DNA, 39 bp, Mycobacterium sp.
cggggcttcttgcactcggcataggcgagtgctaagaat

SEQ ID NO: 24 - DNA, 39 bp, Mycobacterium sp.
tagcggttctagcacttgagacggtagagtgctaacgcc

SEQ ID NO: 25 - DNA, 39 bp, Mycobacterium sp.
cttgagtgctagcactctcatgtatagagtgctagatgg

(19) United States
(12) Patent Application Publication
(10) Pub. No.: US 2004/0219159 A1
(43) Pub. Date: Nov. 4, 2004

(54) Methods and compositions for therapeutic intervention in infectious disease

(76) Inventors: Graham Stewart, Walton-on-Thames (GB); Peadar O'Gaora, London (GB); Douglas Young, Ruislip (GB)

Correspondence Address: JOHN S. PRATT, ESQ, KILPATRICK STOCKTON, LLP, 1100 PEACHTREE STREET, ATLANTA, GA, 30309, US

(21) Appl. No.: 10/807,963

(22) Filed: Mar. 24, 2004

Related U.S. Application Data
(63) Continuation of application No. 10/079,653, filed on Feb. 20, 2002, now abandoned.
(60) Provisional application No. 60/269,801, filed on Feb. 20, 2001.
(60) Provisional application No. 60/294,170, filed on May 29, 2001.

(51) Int. Cl. A61K 39/00; A61K 39/38; A61K 39/04

(52) U.S. Cl. 424/184.1

(57) ABSTRACT

Methods and compositions for the treatment and prevention of infectious diseases are provided. In particular, efficient vaccines comprising genetically modified pathogens are provided. The vaccines generally comprise mycobacterial mutants having modified protein production capabilities. In one embodiment, the mutants overexpress heat shock protein. In a specific embodiment, the mycobacterial mutant overexpresses heat shock proteins 60 and/or 70. Also provided are modified BCG vaccines capable of overexpressing heat shock proteins 60 and/or 70.

### CROSS REFERENCE TO RELATED APPLICATIONS

This application is the non-provisional application of U.S. Provisional Application Ser. No. 60/269,801 filed Feb. 20, 2001, and U.S. Provisional Application Ser. No. 60/294,170 filed May 29, 2001.

### FIELD OF INVENTION

The present invention relates to methods and compositions for treating infectious diseases. In particular, the invention relates to the manipulation of antigen production by infectious organisms. More particularly, the present invention comprises manipulation of mycobacterial genes resulting in the modification of heat shock protein production.

### BACKGROUND OF THE INVENTION

Mycobacterial infections often manifest as diseases such as tuberculosis. Human infections caused by mycobacteria have been widespread since ancient times, and tuberculosis remains a leading cause of death today. Although the incidence of the disease declined in parallel with advancing standards of living since at least the mid-nineteenth century, mycobacterial diseases still constitute a leading cause of morbidity and mortality in countries with limited medical resources and can cause overwhelming, disseminated disease in immunocompromised patients. In spite of the efforts of numerous health organizations worldwide, the eradication of mycobacterial diseases has never been achieved, nor is eradication imminent. Nearly one third of the world's population is infected with M. tuberculosis complex, commonly referred to as tuberculosis (TB), with approximately 8 million new cases and 3 million deaths attributable to TB yearly.

After decades of decline, TB is on the rise. In the United States, up to 15 million individuals are believed to be infected. Almost 28,000 new cases were reported in 1990, a 9.4 percent increase over 1989. A sixteen percent increase was observed from 1985 to 1990. Overcrowded living conditions and shared air spaces are especially conducive to the spread of TB, contributing to the increase in instances that have been observed in the U.S. in prison inmates and among the homeless in larger cities.

Approximately half of all patients with acquired immune deficiency syndrome (AIDS) will acquire a mycobacterial infection, with TB being an especially devastating complication. AIDS patients are at higher risks of developing clinical TB and anti-TB treatment seems to be less effective than in non-AIDS patients. Consequently, the infection often progresses to a fatal disseminated disease.

Mycobacteria other than M. tuberculosis are increasingly found in opportunistic infections that plague the AIDS patient. Organisms from the M. avium-intracellulare complex (MAC), especially serotypes four and eight, account for 68% of the mycobacterial isolates from AIDS patients. Enormous numbers of MAC are found (up to 10¹⁰ acid-fast bacilli per gram of tissue) and, consequently the prognosis for the infected AIDS patient is poor.

Crohn's disease is a chronic inflammatory bowel disease characterized by transmural inflammation and granuloma formation. Mycobacterium avium subspecies paratuberculosis (M. paratuberculosis) causes a similar disease in animals. Johnes's disease, affecting cattle, causes estimated losses of $1.5 billion to the agriculture industry in the US (Clinical Microbiology Reviews July 2001 p 489-512,). Isolation of M. paratuberculosis from intestional tissue of Crohn's disease patients has led to concern that it may be pathogenic for humans. Nevertheless a causal relationship has not been demonstrated.

Cattle also suffer from infection with Mycobacterium bovis which causes a disease similar to tuberculosis. Control of infection is a serious herd management concern. This infection can be transferred to humans.

The World Health Organization (WHO) continues to encourage the battle against TB, recommending prevention initiatives such as the “Expanded Program on Immunization” (EPI), and therapeutic compliance initiatives such as “Directly Observed Treatment Short-Course” (DOTS). For the eradication of TB, diagnosis, treatment, and prevention are equally important. Rapid detection of active TB patients will lead to early treatment by which about 90% cure is expected. Therefore, early diagnosis is critical for the battle against TB. In addition, therapeutic compliance will ensure not only elimination of infection, but also reduction in the emergence of drug-resistance strains.

The emergence of drug-resistant M. tuberculosis is an extremely disturbing phenomenon. The rate of new TB cases proven resistant to at least one standard drug increased from 10 percent in the early 1980's to 23 percent in 1991. Compliance with therapeutic regimens, therefore, is also a crucial component in efforts to eliminate TB and prevent the emergence of drug-resistant strains. Equally important is the development of new therapeutic agents that are effective as vaccines and as treatments for disease caused by drug resistant strains of mycobacteria.

Although over 37 species of mycobacteria have been identified, more than 95% of all human infections are caused by six species of mycobacteria: M. tuberculosis, M. avium-intracellulare, M. kansasii, M. fortuitum, M. chelonae, and M. leprae. The most prevalent mycobacterial disease in humans is tuberculosis (TB) which is caused by mycobacterial species comprising M. tuberculosis, M. bovis, or M. africanum (Merck Manual 1992). Infection is typically initiated by the inhalation of infectious particles which are able to reach the terminal pathways in lungs. Following engulfment by alveolar macrophages, the bacilli are able to replicate freely, with eventual destruction of the phagocytic cells. A cascade effect ensues wherein destruction of the phagocytic cells causes additional macrophages and lymphocytes to migrate to the site of infection, where they too are ultimately eliminated. The disease is further disseminated during the initial stages by the infected macrophages which travel to local lymph nodes, as well as into the blood stream and other tissues such as the bone marrow, spleen, kidneys, bone and central nervous system. (See Murray et al. Medical Microbiology, The C.V. Mosby Company 219-230 (1990)).

There is still no clear understanding of the factors which contribute to the virulence of mycobacteria. Many investigators have implicated lipids of the cell wall and bacterial surface as contributors to colony morphology and virulence. Evidence suggests that C-mycosides, on the surface of certain mycobacterial cells, are important in facilitating survival of the organism within macrophages. Trehalose 6,6′ dimycolate, a cord factor, has been implicated for other mycobacteria.

The interrelationship of colony morphology and virulence is particularly pronounced in M. Avium. M. avium bacilli occur in several distinct colony forms. Bacilli which grow as transparent or rough colonies on conventional laboratory media are able to multiply within macrophages in tissue culture, are virulent when injected into susceptible mice, and are resistant to antibiotics. Rough or transparent bacilli which are maintained on laboratory culture media often spontaneously assume an opaque colony morphology at which time they fail to grow in macrophages, are avirulent in mice, and are highly susceptible to antibiotics. The differences in colony morphology between the transparent, rough and opaque strains of M. avium are almost certainly due to the presence of a glycolipid coating on the surface of transparent and rough organisms which acts as a protective capsule. This capsule, or coating, is composed primarily of C-mycosides which apparently shield the virulent M. avium organisms from lysosomal enzymes and antibiotics. By contrast, the non-virulent opaque forms of M. avium have very little C-mycoside on their surface. Both resistance to antibiotics and resistance to killing by macrophages have been attributed to the glycolipid barrier on the surface of M. avium.

Diagnosis of mycobacterial infection is confirmed by the isolation and identification of the pathogen, although conventional diagnosis is based on sputum smears, chest X-ray examination (CXR), and clinical symptoms. Isolation of mycobacteria on a medium takes as long a time as four to eight weeks. Species identification takes a further two weeks. There are several other techniques for detecting mycobacteria such as the polymerase chain reaction (PCR), mycobacterium tuberculosis direct test, or amplified mycobacterium tuberculosis direct test (MTD), and detection assays that utilize radioactive labels.

One diagnostic test that is widely used for detecting infections caused by M. tuberculosis is the tuberculin skin test. Although numerous versions of the skin test are available, typically one of two preparations of tuberculin antigens are used: old tuberculin (OT), or purified protein derivative (PPD). The antigen preparation is either injected into the skin intradermally, or is topically applied and is then invasively transported into the skin with the use of a multiprong inoculator (Tine test). Several problems exist with the skin test diagnosis method. For example, the Tine test is not generally recommended because the amount of antigen injected into the intradermal layer cannot be accurately controlled. (See Murray et al. Medical Microbiology, The C.V. Mosby Company 219-230 (1990)).

Although tuberculin skin tests are widely used, they typically require 2-3 days to generate results, and many times, the results are inaccurate as false positives are sometimes seen in subjects who have been exposed to mycobacteria but are healthy. In addition, instances of mis-diagnosis are frequent since a positive result is not observed only in active TB patients, but also in BCG-vaccinated persons and those who had been infected with mycobacteria but have not developed the disease. It is hard therefore, to distinguish active TB patients from the others, such as household TB contacts, by the tuberculin skin test. Additionally, the tuberculin test often produces a cross-reaction in those individuals who were infected with mycobacteria other than M. tuberculosis (MOTT). Diagnosis using the skin tests currently available is frequently subject to error and inaccuracies.

The standard treatment for tuberculosis caused by drug-sensitive organisms is a 6-month regimen consisting of four drugs given for 2 months, followed by two drugs given for 4 months. The two most important drugs, given throughout the 6-month course of therapy, are isoniazid and rifampin. Although the regimen is relatively simple, its administration is quite complicated. Daily ingestion of the eight or nine pills often required during the first phase of therapy can be a daunting and confusing prospect. Even severely ill patients are often symptom-free within a few weeks, and nearly all appear to be cured within a few months. If the treatment is not continued to completion, however, the patient may experience a relapse, and the relapse rate for patients who do not continue treatment to completion is high. A variety of forms of patient-centered care are used to promote adherence with therapy. The most effective way of ensuring that patients are taking their medication is to use directly observed therapy, which involves having a member of the health care team observe the patient take each dose of each drug. Directly observed therapy can be provided in the clinic, the patient's residence, or any mutually agreed upon site. Nearly all patients who have tuberculosis caused by drug-sensitive organisms and who complete therapy will be cured, and the risk of relapse is very low. (“Ending Neglect: The Elimination of Tuberculosis in the United States” ed. L. Geiter Committee on the Elimination of Tuberculosis in the United States Division of Health Promotion and Disease Prevention, Institute of Medicine. Unpublished.)

Clearly, a vaccine that would prevent the onset of tuberculosis and therefore eliminate the need for therapy is desirable. Although currently available vaccines such as the BCG are effective, the emergence of drug resistant strains has necessitated new formulations and compositions that are more versatile than the BCG.

What are needed are effective therapeutic regimens that include improved vaccination and treatment protocols. Currently available therapeutics are no longer consistently effective as a result of the problems with treatment compliance contributing to the development of drug resistant mycobacterial strains.

### SUMMARY OF THE INVENTION

The present invention comprises methods and compositions for the treatment of infectious diseases. In accordance with a preferred embodiment of the present invention, methods for the manipulation of infectious organism genes resulting in the modification of protein production are provided. Specifically, the present invention provides a teaching of mycobacterial genetic manipulation which results in an increase in heat shock protein production. The increase in heat shock protein production results in an enhanced immune response to the heat shock proteins and also other mycobacterial proteins in general.

Heat shock proteins (hsp) are widely distributed in nature and are among the most highly conserved molecules of the biosphere. Heat shock proteins perform important functions in the folding and unfolding or translocation of proteins, as well as in the assembly and disassembly of protein complexes. Because of these helper functions, heat shock proteins have been termed molecular chaperones. Heat shock protein synthesis is increased to protect prokaryotic or eukaryotic cells from various insults during periods of stress caused by infection, inflammation, or similar events. (Zugel et al. Clinical Microbiology Reviews 12(1) pp 19-39 (1999)).

The inventors of the present invention provide for the first time a teaching of the use of pathogenic, and more specifically mycobacterial, heat shock proteins in novel vaccines and therapeutics. The findings of the inventors are both unobvious and unexpected since those skilled in the art have not considered the use of heat shock proteins in this capacity. For example, Zugel et al. state that “although hsp play an important role in several infectious and autoimmune diseases, evidence arguing against the direct involvement of heat shock proteins in protection or autoaggression has been gathered. At present, initiation of protective immunity against infectious antigens or autoimmune disorders by heat shock proteins alone appears unlikely.” (Zugel et al. Clinical Microbiology Reviews 12(1) pp 19-39 (1999) (emphasis added)).

Unlike prior art methods, the treatment methods and compositions provided herein are highly effective and specific. Most importantly, the treatment methods and compositions of the present invention are especially effective in conferring immunity against M. tuberculosis infection and therefore represent promising candidates for use as new vaccinations.

The vaccination methods described herein involve the manipulation of mycobacterial protein production. Such proteins include, but are not limited to, mycobacterial heat shock proteins such as heat shock protein 60 (Hsp60) (GroEL1, Rv3417c: GroEL2, Rv0440), Hsp10 (GroES, Rv3418c), Hsp70 (Rv0350), DnaJ (Hsp40, Rv0352), GrpE (Rv0351) and ClpB (Rv0384c) and Hsp90. A particularly preferred embodiment of the invention comprises a mutant strain of M. tuberculosis that constitutively overexpresses Hsp70. Another preferred embodiment of the present invention comprises M. bovis BCG (hereafter ‘BCG’) vaccines capable of heat shock protein overexpression. In another preferred embodiment, mutant strains of mycobacteria or BCG overexpress more than one heat shock protein; such mutants include for example, strains that overexpress both Hsp70 and Hsp60. The present invention contemplates other combinations of heat shock protein overexpression. The present invention further contemplates overexpression of other mycobacterial proteins such as antigenic proteins found in the cell wall or secreted by the pathogen.

Accordingly, it is an object of the present invention to provide methods and compositions for the treatment and prevention of infectious diseases.

Another object of the present invention is to provide methods and compositions for the treatment and prevention of mycobacterial disease such as tuberculosis.

It is another object of the present invention to provide methods and compositions for the treatment and prevention of mycobacterial disease using compositions comprising genetically altered mycobacteria that are capable of overexpressesing certain proteins.

Another object of the present invention is to provide methods and compositions for the treatment and prevention of tuberculosis using compositions comprising genetically altered mycobacteria that overexpress certain proteins, wherein the proteins comprise heat shock proteins, cell wall proteins or other antigenic proteins secreted by the pathogen.

Yet another object of the present invention is to provide methods and compositions for the treatment and prevention of tuberculosis wherein the proteins overexpressed by the genetically altered mycobacteria comprise Hsp60, Hsp70 and various combinations thereof.

Another object of the present invention is to provide compositions for vaccine formulations for the prevention of mycobacterial disease.

Another object of the present invention is to provide compositions which alert, stimulate and direct the immune response to a more protective state.

Yet another object of the present invention is to provide compositions for vaccine formulations for the prevention of mycobacterial disease caused by mycobacterial species comprising M tuberculosis complex, M. avium-intracellulare, M. kansasii, M. fortuitum, M. chelonae, M. leprae, M. afticanum, and M. microti and M. paratuberculosis

Another object of the present invention is to provide methods for the manipulation of pathogenic organisms, namely mycobacterial genes, resulting in the modification of protein production.

It is yet another object of the present invention to provide methods and compositions for production of mycobacterial mutants characterized by a defective heat shock response.

Another object of the present invention is to provide methods and compositions for production of mycobacterial mutants wherein the hspR gene of M. tuberculosis has been modified resulting in the overexpression of Hsp70.

Another object of the present invention is to provide methods and compositions for production of mycobacterial mutants wherein the hspR gene of BCG has been modified resulting in the overexpression of Hsp70.

Another object of the present invention is to provide methods and compositions for production of mycobacterial mutants wherein the hrcA gene of M. tuberculosis has been modified resulting in the overexpression of Hsp60.

It is another object of the present invention to provide methods and compositions for production of mycobacterial mutants wherein the hrcA gene of M. bovis has been modified resulting in the overexpression of Hsp60.

Yet another object of the present invention is to provide methods and compositions for production of mycobacterial mutants wherein both the hspR and hrcA genes of M. tuberculosis have been modified resulting in the overexpression of both Hsp70, Hsp60 and co-regulated proteins.

Another object of the present invention is to provide methods and compositions for production of mycobacterial mutants wherein both the hspR and hrcA genes of BCG have been modified resulting in the overexpression of both Hsp70, Hsp60 and co-regulated proteins

Another object of the present invention is to provide a counterselectable suicide vector for gene replacement of hrcA in M. tuberculosis and BCG.

These and other objects, features and advantages of the present invention will become apparent after a review of the following detailed description of the disclosed embodiments and the appended claims.

### BRIEF DESCRIPTION OF THE FIGURES

FIG. 1. Structure, regulation and mutagenesis of the hsp70 (dnaK) operon.

a. The hsp70 operon comprises four open reading frames, preceded by two copies of the HAIR (HspR Associated Inverted Repeat) element (HAIR1, 5′-CTTGAGCGGGGTGCACTCATC-3′ (SEQ ID NO: 1) and HAIR2,5′-GTTGAGTGCATCAGGCTCAGC-3′; (SEQ ID NO: 2) identity to the consensus HAIR, 5′-CTTGAGT-N-7-ACTCAAG-3′ (SEQ ID NO: 3), is underlined). TSP1 and TSP2 indicate transcriptional start points.

b. Gel shift analysis of interactions between histidine-tagged recombinant HspR and a double-stranded oligonucleotide corresponding to the HAIR element. *HspR-HAIR complex. **Temperature-sensitive super-shifted band.

c. Southern blot analysis of Pst1 digested genomic DNA probed with the HS1/HS2 PCR product corresponding to grpE and dnaJ. Lane 1, λ HindIII ladder; lane 2, M. tuberculosis H37Rv; lane 3, M. tuberculosis hspR mutant.

FIG. 2. Constitutive overexpression of hsp70 proteins in the HspR mutant.

a. Mapping of transcriptional start points for the hsp70 operon using mRNA extracted from wild type BCG (WT) and the ΔhspR mutant with and without heat shock.

b. SDS-PAGE analysis of [³⁵ S]-methionine-labeled proteins from wild type BCG (WT) and the ΔhspR mutant with and without heat shock.

FIG. 3. Growth and survival of the ΔhspR mutant in stationary phase, heat stress conditions and macrophages.

The ΔhspR mutant (v) was compared to wild type M. tuberculosis (◯) with respect to growth in laboratory culture.

a. exponential growth and stationary phase survival in liquid broth.

b. survival at high temperature.

Mutant and wild type strains were compared for growth and survival in bone marrow derived macrophages.

c. growth in quiescent macrophages.

d. survival in activated macrophages.

Error bars show±SE.

FIG. 4. Characterization of the ΔhspR mutant in a chronic infection model.

Mice were infected with wild type M. tuberculosis (◯) and the corresponding ΔhspR mutant (v) and the bacterial load assessed in homogenised lung and spleen tissues. Bacterial load in the spleen (a) and lung (b) during the chronic phase of infection. Each data point represents the mean of four replicates in a single representative experiment. Error bars show±SE. (c) Bacterial load in the lung during acute infection. Data points represent mean values from three independent experiments each with at least three replicates per time point.

FIG. 5. Lung morphology in mice infected with wild type and mutant strains.

Histological examination of representative sections from the lungs of mice 14 weeks after infection with the ΔhspR mutant (a) and wild type M. tuberculosis (b). Magnification, ×1000.

FIG. 6. Infection with the ΔhspR mutant increases IFN-γ production by splenocytes.

Mice were infected with BCG ΔhspR (v) and wild-type BCG (◯) and the immune response in splenocytes was analysed by ELISPOT and flow cytometry.

a. IFN-γ ELISPOT of Hsp70-stimulated cells.

b. Ratio of Hsp70-specific IFN-γ to IL-4 producing cells.

c. Intracellular IFN-γ production in CD4⁺ and CD8⁺ T cells. Data from day 35 post-infection.

Each symbol represents an individual mouse.

FIG. 7. Counterselectable suicide vector for gene replacement of hrcA in M. tuberculosis and BCG

FIG. 8. Southern blot of Kpn1 digested gDNA probed with HRCA1/HRCA2. Lane 1, hindIII digest of λ DNA; lane2, M. tuberculosis ΔhspR; lane 3, M. tuberculosis ΔhspR ΔhrcA

FIG. 9. SDS-PAGE showing overexpressed ClpB, Hsp70, Hsp60 and Hsp10 (GroES) in the hspR and hrcA deleted strain. Lane 1, wild type M. tuberculosis H37Rv; lane 2, M. tuberculosis ΔhspR ΔhrcA

FIG. 10. Gene expression profiles of M. tuberculosis during heat shock and of M. tuberculosis lacking the transcriptional repressor, HspR. Scatter plots show log Cy5/Cy3 signal ratios against log total signal intensity where log ratios are centralised such that mean log Cy5 and Cy3 are equal to zero. A, Expression of M. tuberculosis genes at 45° C. (Cy5) versus 37° C. (Cy3). B, Expression in M. tuberculosis ΔhspR (Cy5) versus wild-type M. tuberculosis H37Rv (Cy3) at 37° C. C, Expression in M. tuberculosis ΔhspR complemented with a functional copy of hspR on the integrating plasmid pSMT168 (Cy5) versus wild-type M tuberculosis H37Rv (Cy3) at 37° C.

FIG. 11. Functional distribution of genes upregulated during heat shock. Frequency of genes among functional groups (http://genolist.pasteur.fr/TubercuList/) across the genome (grey bars) and among heat shock induced genes (black bars).

FIG. 12. Heat shock repressor binding sites within M. tuberculosis. A, HspR associated inverted repeat or HAIR sequences. B, HrcA binding sites or CIRCE (controlling inverted repeat of chaperone expression).

FIG. 13. Deletion of hrcA and hspR results in overexpression of Hsp70 (DnaK), Hsp60 (GroEL), Hsp10 (GroES) and a protein consistent in size with Acr2. A, Southern blot of Kpn1 digested genomic DNA demonstrating deletion of hrcA in M. tuberculosis ΔhspR. Lane 1, HindIII digested X DNA; lane 2, M. tuberculosis ΔhspR (3634 bp wild-type hrcA hybridising fragent); lane 3, M. tuberculosis ΔhspRΔhrcA (6526 bp hrcA-deleted fragment). B, Protein extracts of 37° C. cultured M. tuberculosis H37Rv (lane 1) and M. tuberculosis ΔhspRΔhrcA (lane 2) separated by SDS-PAGE and stained with coomassie brilliant blue.

FIG. 14. Table 1. Upregulated genes in M. tuberculosis ΔhspR compared to wild-type H37Rv. cDNAs from the mutant and wild-type strains were labelled with Cy5 and Cy3 respectively and competitively hybridised to a complete genome DNA microarray. Relative signal intensities of Cy3 and Cy5 were assessed by an ANOVA and upregulation considered significant where p<0.01. The mean fold change in gene expression is also shown alongside the mean fold change in expression of a complemented mutant strain also compared to wild-type. CH=conserved hypothetical protein.

FIG. 15. Table 2 Upregulated genes in M. tuberculosis ΔhspRΔhrcA compared to wild-type H37Rv. cDNAs from the mutant and wild-type strain were labelled with Cy5 and Cy3 respectively and competitively hybridised to a complete genome DNA microarray. Relative signal intensities of Cy3 and Cy5 were assessed by an ANOVA and upregulation considered significant where p<0.01. CH=conserved hypothetical protein.

### DETAILED DESCRIPTION

The present invention may be understood more readily by reference to the following detailed description of specific embodiments included herein. Although the present invention has been described with reference to specific details of certain embodiments thereof, it is not intended that such details should be regarded as limitations upon the scope of the invention.

The entire text of the references mentioned herein are hereby incorporated in their entireties by reference, including U.S. Provisional Application Ser. No. 60/269,801 filed Feb. 20, 2001, and U.S. Provisional Application Ser. No. 60/294,170 filed May 29, 2001.

Mycobacterial infections such as those causing tuberculosis, once thought to be declining in occurrence, have rebounded and again constitute a serious health threat. Areas where humans are crowded together or living in substandard housing are increasingly found to have persons infected with mycobacteria. Persons who are immunocompromised are at great risk of being infected with mycobacteria and dying from such infection. In addition, the emergence of drug-resistant strains of mycobacteria has added to the treatment problems of such infected persons.

Many people who are infected with mycobacteria are poor or live in areas with inadequate health care facilities. As a result of various obstacles (economical, education levels etc.) many of these individuals are unable to comply with prescribed therapeutic regimens, and ultimately persistent non-compliance by these and other individuals results in the prevalence of disease frequently compounded by the emergence of drug resistant strains of mycobacteria. Effective vaccines that target various strains of mycobacteria are necessary to bring the increasing numbers of tuberculosis under control.

The present invention provides methods and compositions comprising genetically modified pathogenic organisms such as mycobacteria for the prevention and treatment of infectious disease such as tuberculosis. More particularly, the present invention provides mycobacterial mutants capable of altered protein expression. As described herein, the protein that has altered expression may be overexpressed and may comprise any relevant mycobacterial protein, such as a cell wall protein or other antigenic protein secreted by the pathogen. Typically, the overexpressed protein is a heat shock protein such as Hsp60 or Hsp70. In an alternative embodiment of the present invention, ‘multiple’ mutants i.e. genetically modified mycobacteria capable of altered expression of more than one protein, are also provided. In a particular embodiment, ‘double’ mutants capable of overexpressing Hsp60 and Hsp70 related proteins, are provided.

In addition to the above-described embodiments, the present invention also provides improved BCG vaccines capable of overexpressing heat shock proteins. In a most preferred embodiment, a vaccine comprising BCG capable of overexpressing both Hsp60 and Hsp 70 and co-regulated proteins is provided.

The methods and compositions of the present invention may be used for vaccinating and treating mycobacteria infection in humans as well as other animals. For example, the present invention may be particularly useful for the prevention of disease in cows infected by M. bovis.

As used herein the term “tuberculosis” comprises disease states usually associated with infections caused by mycobacteria species comprising M. tuberculosis complex. Mycobacterial infections caused by mycobacteria other than M. tuberculosis (MOTT) are usually caused by mycobacterial species comprising M. avium-intracellulare, M. kansasii, M. fortuitum, M. chelonae, M. leprae, M. africanum, M. microti and M. paratuberculosis.

Elevated expression of heat shock proteins can benefit a microbial pathogen struggling to penetrate host defenses during infection, but at the same time may provide a crucial signal alerting the host immune system to its presence. To determine which of these effects predominate, the present inventors constructed a mutant strain of M. tuberculosis that constitutively overexpresses Hsp70 proteins. Surprisingly, although the mutant was fully virulent in the initial stage of infection, it was significantly impaired-in its ability to persist during the subsequent chronic phase. As demonstrated herein, the present inventors discovered that induction of microbial heat shock genes provides a novel strategy to boost the immune response of individuals harboring latent tuberculosis infection.

Cells exposed to elevated temperature or other stress stimuli respond by increased expression of heat shock proteins.¹ The heat shock response, and the proteins involved, have been highly conserved throughout evolution from Escherichia coli to man. The major heat shock proteins are molecular chaperones with an essential role in directing folding and assembly of polypeptides within the cell.² Enhanced expression of heat shock proteins in response to stress allows cells to tolerate potentially harmful consequences associated with intracellular accumulation of denatured polypeptides.

Synthesis of heat shock proteins is induced in microbial pathogens during infection³⁻⁵. While the increased level of these proteins is likely to enhance microbial survival in the face of attack by host immune cells, the present inventors have discovered that it may also provide an important signal in alerting the host to the presence of the pathogen. Heat shock proteins interact with the immune system through a variety of mechanisms. They were initially identified as prominent antigens in a range of infectious diseases and autoimmune disorders^{6,7}. In addition to immune recognition of the proteins themselves, their functional role as chaperones is associated with an ability to promote immune responses to other polypeptides^{8,9}. Finally, although the functional role of heat shock proteins is primarily intracellular, several studies suggest that exogenous heat shock proteins trigger immunomodulatory signals as a result of recognition by cell surface receptors^{10-12.}

Current knowledge in this area provides that heat shock proteins are mainly associated with disease and that these proteins are “virulence factors” that constitute the part of the mycobacterial organism that is fundamentally responsible for disease. Contrary to current knowledge however, the present inventors have examined the role of these possible virulence factors and surprisingly found, that when overexpressed, the resulting pathogenic state did not advance as fast-as the wild type, instead it stimulated the immune system more than wildtype and caused less pathology. Accordingly, another important aspect of the present invention is that overexpression of the mycobacterial heat shock protein not only increases the immune response to that particular protein, but it also enhances the immune response to other mycobacterial proteins.

The present study was designed to explore the apparent paradox that increased expression of heat shock proteins has the potential to benefit both the pathogen and the host during infection. The inventors focused on M. tuberculosis, a pathogen characterized by an intimate and prolonged interaction with the host immune system. M. tuberculosis has adapted to survival within the toxic environment of phagocytic cells, with the outcome of infection crucially dependent on the host cell-mediated immune response. Heat shock proteins were amongst the first antigens identified from M. tuberculosis⁷, and are currently under investigation as vaccine candidates¹⁴. The present experimental strategy was firstly to investigate the genetic basis of heat shock regulation in M. tuberculosis, and then to construct a mutant strain with a defective heat shock response. As described herein, the inventors have created novel M. tuberculosis mutants characterized by constitutive overexpression of Hsp70, and/or Hsp60, and related proteins, and demonstrated that this ultimately results in a bias in favor of the host rather than the pathogen during chronic infection.

Although mycobacterial heat shock proteins have been used extensively in immunological experiments, relatively little attention has been given to regulation of the mycobacterial heat shock response. As detailed in the Examples section, the present inventors have demonstrated that Hsp70 expression in M. tuberculosis is regulated by a repressor system analogous to that in Streptomyces²⁴. The HspR repressor controls expression of only a small number of genes in M. tuberculosis, comprising the hsp70 operon and the gene encoding the ATPase ClpB^{23,28}, which like Hsp70 is preceded by an inverted repeat resembling the HAIR element.

In contrast to the toxic effect of Hsp70 overexpression in E. coli²⁹, constitutive overexpression of the Hsp70 proteins resulted in only a slightly reduced growth rate of M. tuberculosis under in vitro culture conditions. This is consistent with the relatively modest effect of hspR deletion on the in vitro phenotype of Streptomyces mutants³⁰ and is presumably due to the extra metabolic load of increased protein production. Increased thermotolerance of M. tuberculosis ΔhspR is consistent with the proposed function of Hsp70 proteins in response to stress. In contrast, overexpression of heat shock proteins in E. coli was not on its own sufficient to increase thermotolerance³¹.

The phenotype of the ΔhspR mutant during murine infection is of considerable interest. The availability of tools for mycobacterial mutagenesis has allowed identification of a number of genes involved in virulence of M. tuberculosis. Most of these mutations result in defects in macrophage survival and during the acute phase of infection³²⁻³⁴. Two loci resemble hspR in generating mutants with defects specifically affecting the chronic, or persistent, phase of infection. Mutation in a cyclopropane synthetase gene interferes with lipid biosynthesis causing a change in the surface structure of the mycobacteria and affecting survival in the chronic phase³⁵. Deletion of the gene encoding the enzyme isocitrate lyase similarly reduces persistence³⁶. A probable explanation in this case is that utilization of fatty acid derived substrates via the glyoxylate pathway makes an essential contribution to mycobacterial metabolism in the chronic phase of infection.

Though not wishing to be bound by the following theory, two general mechanisms can be proposed to account for reduced survival of the ΔhspR mutant. Firstly, the high level of the Hsp70 proteins within the cell may block some developmental program involved in mycobacterial adaptation. If, for example, persistence involves formation of some spore-like ‘dormant’ form of the organism³⁷, it is possible that this pathway is blocked in the mutant. While this mechanism cannot be excluded, the enhanced thermotolerance and the absence of any stationary phase defect suggest that the mutant is unimpaired for survival under conditions inimical to replication. Survival in activated macrophages indicates that, in contrast to the isocitrate lyase mutant³⁶, the ΔhspR mutant is able to undergo metabolic adaptation required for survival in an acidified intracellular compartment.

In an alternative and preferred theory, the present inventors propose that the ΔhspR phenotype is immune mediated. This is consistent with the fact that it is evident only after the onset of the acquired immune response. There are several mechanisms by which increased expression of Hsp70 might enhance immune recognition of the ΔhspR mutant. By increasing the antigen load per bacterium, Hsp70 overexpression may either prime a stronger immune response or make cells infected by the mutant more attractive targets for effector immune mechanisms. Regardless of the mechanisms, the present inventors have successfully demonstrated an enhanced immune response as a result of exposure to the ΔhspR mutant. Specifically the inventors have surprisingly shown that infection of mice with BCG ΔhspR induces an increased number of Hsp70-specific IFN-γ secreting splenocytes in comparison to wild type BCG. The enhanced immune response observed under these conditions, presents mycobacterial mutants capable of overexpressing heat shock proteins as excellent candidates for use in novel vaccines and treatments for tuberculosis.¹

In addition to recognition of the Hsp70 protein itself, the chaperone function of Hsp70 presents further potential for immune enhancement. Although enhanced secretory production of a single-chain antibody fragment by coproduction of molecular chaperones has been observed in Bacillus subtilis,³⁸ constitutive overexpression of heat shock proteins in mycobacteria resulting in enhanced immune response has been demonstrated for the first time by the present inventors. Secretion of proteins from viable mycobacteria is thought to facilitate their early immune recognition and is used as a criterion for selection of candidate antigens for inclusion in subunit vaccines³⁹. The findings of the present inventors demonstrate that the effect of Hsp70 overexpression on protein secretion in vivo enhances immune responses to other mycobacterial proteins. Hsp70 released from mycobacterial cells promotes presentation of mycobacterial antigens or antigen fragments attached to its peptide-binding site. Consistent with both of the above scenarios, infection of mice with BCG ΔhspR induced an increased number of CD8⁺ IFN-γ secreting T cells in the spleen. The increase in Hsp70-specific IFN-γ producing cells was not in itself sufficient to account for this difference; there must be some other additional enhancement of CD8⁺ IFN-γ responses and the enhanced immune response is attributed to the chaperone function of Hsp70.

Accordingly, the enhanced immune response observed following exposure to mycobacterial mutants overexpressing heat shock proteins is not solely a result of the increase in the amount of heat shock proteins present themselves, it is also thought to be a result of the chaperone function of the heat shock protein. Therefore, functions of proteins such as Hsp70 in promoting the secretion of other mycobacterial proteins, promoting the immune presentation of other mycobacterial antigens and acting directly on immune cells inducing accessory immune signals, are also important characteristics of any heat shock protein overexpressing strain.

While further analysis of the hspR mutant provides an opportunity to assess these different aspects of heat shock protein immunogenicity, the present study demonstrates that, on balance, Hsp70 overexpression favors the host over the pathogen during the chronic phase of tuberculosis infection. With an estimated one third of the global population currently infected with M. tuberculosis⁴¹, interventions targeted against persistent mycobacteria could have profound public health impact. Induction of mycobacterial heat shock protein expression by specific disruption of HspR regulation or by promotion of protein denaturation, for example may provide a novel strategy for reinforcement of host defenses during latent tuberculosis.

Microarray analysis of an hspR deletion mutant of M. tuberculosis confirms and extends the above-described studies of Hsp70 regulation. HspR is a DNA-binding protein related to the MerR family. It recognises either of two inverted repeat sequences (HAIR) in the promoter region of the hsp70 operon, reducing the level of transcription in unstressed conditions. The HspR protein interacts tightly with Hsp70 in vitro.^{47,68} A system where this heterodimer forms the functional repressor unit with feedback achieved by titration of Hsp70 away from the HspR complex in the presence of unfolded polypeptides represents an attractive model for regulation.^{10,63} We show that in the absence of HspR there is release of transcriptional repression and the genes of the Hsp70 operon are upregulated. Surprisingly, there were also a further 46 genes with significantly elevated transcription. Of these, only three genes (Rv0249c-Rv0251c), arranged consecutively in an apparent operon, were associated with a HAIR-like sequence. Interestingly, the lead gene Rv0251c has also been shown to be under the control of the heat-shock responsive ECF sigma factor, σE, and is also prominent in response to treatment with SDS.⁶¹ This dual control mechanism may account for the relatively modest elevation of Rv0251c transcription in the ΔhspR mutant compared to that observed under heat shock conditions in the wild-type.

Rv0251c encodes a 159 amino acid protein belonging to the small heat shock protein family, termed Hsp20, or the α-crystallin family. Its predicted size is consistent with the approximately 20 kD protein observed by SDS-PAGE to be upregulated in the ΔhspRΔhrcA mutant (FIG. 12B). The small heat shock proteins, like the larger heat shock protein families, are found widely in bacterial and eukaryotic cells and appear to function as molecular chaperones at least in vitro.^{49,75} There are two members of this family in M tuberculosis. The other family member was originally identified as a prominent antigen and is variously referred to as the 14 kD antigen, 16 kD antigen, Hsp16.3, α-crystallin (Acr), or HspX. This gene is not induced by heat shock, but is upregulated in stationary phase cultures and during the hypoxic response.^{51,67,77,78} It is possible that the different α-crystallin homologues fulfil analogous functional roles in response to different stresses. The Acr gene is induced following phagocytosis of M. tuberculosis⁶² and is required for growth in macrophages.⁷⁸ It will be of interest to determine whether the protein encoded by Rv0251c, which we term Acr2, also plays a role during infection.

Within the ΔhspR-upregulated ORF set, the Hsp70 and Acr2 operon genes were upregulated during heat shock along with bfrB, groES and Rv3654c. The bacterioferritin gene, bfrB, and Rv3654c, encoding an 8 kD protein with unknown function, are not preceded by obvious HspR binding sites, but their coregulation with HAIR-associated genes in both heat shock and the mutant suggest an indirect link to HspR. The majority of genes upregulated in the mutant were neither associated with HAIR sequences nor were they upregulated during heat shock. We conclude that the induction of these genes is a consequence of the physiological changes associated with overexpression of the HspR-regulated proteins and may not be directly relevant to the normal heat shock response. An interesting example of this was the trend for upregulation of ribosomal protein expression, which was also mirrored in the ΔhspRΔhrcA strain.

A surprising omission from the ΔhspR upregulated list was clpB, which encodes another probable molecular chaperone. We have previously shown the elevation of ClpB expression in the mutant by proteomic analysis⁶⁸ which suggests that the clpB mRNA is of a sufficiently short half life to preclude detection of the ΔhspR-associated transcriptional increase. The detection of substantially increased clpB mRNA in the wild-type after heat shock at 45° C. is explained by upregulation of clpB transcription by the heat inducible sigma factor, σH, as well as release of HspR repression.⁶⁶

Though not wishing to be bound by the following theory, it is thought that release of HspR repression significantly influences heat shock protein production and may therefore have a corresponding effect on the host immune system. The findings of heat shock protein manipulation are not limited to mycobacterial organisms, and may also be extrapolated to other infectious agents that express heat shock protein.

Double and Multiple Mutants

In order to create mutants having altered expression of more than one mycobacterial protein a similar strategy as discussed above was employed to replace the hrcA gene (Rv2374c) in the ΔhspR strains with the kanamycin resistance gene from Tn903 (kan). The plasmid pSMT99 contains an E. coli origin of replication, the kan gene and the counterselectable marker sacB. The region of DNA upstream of hrcA was amplified by PCR using HF Expand polymerase mix (Roche) and the primers HRCA1 (cgggatccctgttcagtcagcacaccct) (SEQ ID NO: 4) and HRCA2 (gctctagatgtggccgacgagactccca) (SEQ ID NO: 5). The amplification product was digested with xba1 and BamH1 and cloned into BamH1/spe1 digested pSMT99 to make pSMT161. The region of DNA downstream of hrcA was amplified using the primers HRCA3 (gaagatctatgaacgcgcacctgctgca) (SEQ ID NO: 6) and HRCA5 (gaagatctatatccacaatccgctcggt), (SEQ ID NO: 7) cut with BglII and cloned into Bcl1 cut pSMT161 to make pSMT163 (FIG. 7). 1 μg of plasmid was irradiated with 100 mj/cm² UV and electroporated into M. tuberculosis ΔhspR or BCG ΔhspR. Transformants resulting from double crossover integration of the kan gene were selected on 7H11/OADC medium containing 15 μg/ml kanamycin and 2% sucrose. Gene replacement transformants were confirmed by Southern blot, probing Kpn1 digested genomic DNA with digoxigenin labelled HRCA1/HRCA2 PCR product. Wild type strains gave a hybridizing band of approximately 3600 bp and gene replacement strains gave a band of approximately 6500 bp (FIG. 8). Overexpression of Hsp60 and Hsp70 associated proteins was confirmed by SDS-PAGE and coomassie staining of protein extracts from bacteria grown at 37° C. in Middlebrook 7H9 broth (FIG. 9).

Unmarked ΔhspR ΔhrcA strains will be generated using suicide plasmids containing the mutated but unmarked target gene, hyg, sacB and LacZ. The plasmid will be introduced to the mycobacteria as described above and single cross-over integrants selected as hygromycin resistant (hygR), LacZ+ (blue) colonies on hygromycin/X-gal medium. A single clone will be grown in broth and further selected on medium containing 2% sucrose and X-gal for double crossover integration of the mutated target gene. Sucrose resistant, LacZ− (white) colonies will be screened by Southern blot to confirm those derived by gene replacement.

We were able to delete the proposed hrcA gene in the ΔhspR mutant but the same approach has been unsuccessful with wild-type M. tuberculosis. This may reflect some technical problem, but it is also possible that overexpression of Hsp70 proteins compensates in some way for a deleterious effect of hrcA deletion. Upregulation of the major HspR-regulated genes was preserved in the double mutant, alongside upregulation of the HrcA regulon, which included the Hsp60 family genes, groES, groEL1 and groEL2. GroES is functionally related to GroEL and its gene is situated immediately upstream of groEL1. While the expression of groES was enhanced in the ΔhspR mutant, its upregulation in the ΔhspRΔhrcA strain was much greater. The M tuberculosis HrcA protein has yet to be analysed for DNA-binding in vitro, but it has strong sequence similarity to B. subtilis HrcA, and analogous CIRCE-like structures are present in the groES/groEL1 and groEL2 promoter regions. Thus, we can conclude that the HrcA repressor acts as the main transcriptional controller of the Hsp60/GroE family heat shock response, with some cross-talk between the Hsp60 and Hsp70 responses demonstrated by the induction of GroES expression in the hspR deleted strain. The mechanism for this cross-talk is unclear although a weak match for the HspR binding site, HAIR, is present at the beginning of the GroES ORF. Interaction of HspR with this inverted repeat could conceivably have a more subtle effect on transcription than that observed with HAIR sequences that directly overlap the RNA polymerase footprint.

A good match for the CIRCE sequence is found upstream of another ΔhspRΔhrcA upregulated gene, Rv0991c, which encodes a conserved hypothetical protein with unknown function. Expression of both Rv0991c and the adjacent downstream ORF, Rv0990c, was elevated during heat shock but Rv0990c was not significantly upregulated in the mutant. Whether the two genes are transcribed as a bicistronic message or are separately regulated and transcribed remains to be conclusively determined. Thus, it is clear that HrcA regulates not just the Hsp60 heat shock response but also Rv0991c and probably Rv0990c. In light of the effect of the ΔhspR mutation on the virulence of M. tuberculosis⁶⁸, it will be of considerable interest to study the double mutant in infection models.

Based on these studies and the 45° C. transcriptional snapshot, one skilled in the art may conclude that that the HspR and HrcA regulons, which dominate the heat shock proteome comprise only a part of the overall adaptive response. Genes regulated by σH and σE are prominent in the 45° C. response, and upregulation of the σB gene suggests overlap with the general stress response. These different regulatory layers are interlinked, with hsp70 and clpB under dual HspR and σH control, and acr2 under dual HspR and σE control. Moreover, the heat inducible expression of σB and σE is dependent on σH which autoregulates its own expression⁶⁶. In addition, it is probable that the functional activity of the sigma factors is subject to post-translational control by anti-sigma factor pathways.⁵⁷ Detailed analysis of bacteria exposed to different temperatures for different time periods will be important in further dissection of this complex pattern of regulatory circuits.

Techniques similar to those described above may be employed to create mutants continuing multiple modifications resulting in the overexpression of more than one or two heat shock proteins.

Formulations

Therapeutics including vaccines comprising mycobacterial mutants of the present invention, such as BCG overexpressing Hsp60 and/or Hsp70, can be prepared in physiologically acceptable formulations, such as in pharmaceutically acceptable carriers, using known techniques. For example, the mutant is combined with a pharmaceutically acceptable excipient to form a therapeutic composition.

The compositions of the present invention may be administered in the form of a solid, liquid or aerosol. Examples of solid compositions include pills, creams, and implantable dosage units. Pills may be administered orally. Therapeutic creams may be administered topically. Implantable dosage units may be administered locally, for example, in the lungs, or may be implanted for systematic release of the therapeutic composition, for example, subcutaneously. Examples of liquid compositions include formulations adapted for injection intramuscularly, subcutaneously, intravenously, intra-arterially, and formulations for topical (transdermal) and intraocular administration. Examples of aerosol formulations include inhaler formulations for administration to the lungs.

The compositions may be administered by standard routes of administration. In general, the composition may be administered by topical, oral, rectal, nasal or parenteral (for example, intravenous, subcutaneous, or intramuscular) routes. In addition, the composition may be incorporated into sustained release matrices such as biodegradable polymers, the polymers being implanted in the vicinity of where delivery is desired, for example, at the site of a lesion. The method includes administration of a single dose, administration of repeated doses at predetermined time intervals, and sustained administration for a predetermined period of time.

A sustained release matrix, as used herein, is a matrix made of materials, usually polymers which are degradable by enzymatic or acid/base hydrolysis or by dissolution. Once inserted into the body, the matrix is acted upon by enzymes and body fluids. The sustained release matrix desirably is chosen by biocompatible materials such as liposomes, polylactides (polylactide acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxylic acids, fatty acids, phospholipids, polysaccharides, nucleic acids, polyamino acids, amino acids such phenylalanine, tyrosine, isoleucine, polynucleotides, polyvinyl propylene, polyvinylpyrrolidone and silicone. A preferred biodegradable matrix is a matrix of one of either polylactide, polyglycolide, or polylactide co-glycolide (co-polymers of lactic acid and glycolic acid).

The dosage of the composition will depend on the condition being treated, the particular composition used, and other clinical factors such as weight and condition of the patient, and the route of administration.

The composition may be administered in combination with other compositions and procedures for the treatment of other disorders occurring in combination with mycobacterial disease. For example, tuberculosis frequently occurs as a secondary complication associated with acquired immunodeficiency syndrome (AIDS). Patients undergoing treatment AIDS including procedures such as surgery, radiation or chemotherapy may benefit from the therapeutic methods and compositions described herein.

This invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention.

### EXAMPLES

### Example 1

### Characterisation of HspR

The hspR gene from M. tuberculosis was amplified by PCR from pY3111⁴² and ligated into pQE30 (Qiagen, West Sussex, U.K). Transformants in E. coli SG13009 were induced with 2 mM IPTG. Bacteria were lysed in 20 ml 8 M urea in 50 mM Tris-buffered saline pH 8 (TBS), and cleared lysate added to nickel-nitrilo-tri-acetic acid resin in 8 M urea-TBS for 1 hour. After washing with 8 M urea-TBS, bound protein was renatured using a gradient from 6 M urea in TBS to TBS alone, and histidine-tagged HspR eluted with 250 mM imidazole in TBS.

Binding of purified HspR to HAIR2 was tested in a gel shift assay using an α[³²P]-labelled double stranded oligonucleotide generated by annealing DNAKIR-F (5′-GCTCAGTAAGTTGAGTGCATCAGGCTCAGCTCTGAATTG A-3′) (SEQ ID NO: 8) and DNAKIR-R (5′-GTCAATTCAGAGCTGAGCCTGATGCACTCAA CTTACTGA G-3′) (SEQ ID NO: 9). Binding reactions were carried out at 30° C. or 48° C. for 90 minutes in 20 mM HEPES (pH7.9), 20 mM NaCl, 2 mM MgCl₂, 1 mM DTT, 1 mM PMSF, 20 μg BSA, 2 μg sonicated salmon sperm DNA, 20% glycerol, 300 pg labelled oligonucleotide and 150 ng His-tagged HspR with or without 10 μg BCG sonicated cell extract. Products were electrophoresed in 6% native polyacrylamide and migration visualised by autoradiography.

### Example 2

### Generation and Characterization of ΔhspR Mutants

DNA fragments (2 kb) immediately upstream and downstream of hspR were amplified with Pwo polymerase using the primer pairs HS1 (5′-GGACTAGTCGTTGTGGACGCGGAGGTG-3′) (SEQ ID NO: 10)/HS2 (5′-GCTCTAGACCCCGTCCTTTGGGTTCTTC-3′) (SEQ ID NO: 11) and HS3 (5′-GGACTAGTCACCGCCCTGGTC GTCTGG-3′) (SEQ ID NO: 12)/HS4 (5′-GCTCTAGATCAGTGGCACCGTCTTGGC-3′) (SEQ ID NO: 13).

Fragments were cloned into the suicide vector pSMT100 flanking a hygromycin resistance gene, and gene replacement transformants were selected as described previously⁴³. In attempts to restore the wild type phenotype, the cloned hspR gene was reintroduced into M. tuberculosis on plasmid vectors under the control of the constitutively active superoxide dismutase (sodA) promoter, or the inducible M. smegmatis acetamidase promoter using vectors pSODIT-2 and pACE-5 respectively⁴⁴.

Stationary phase survival was monitored in 100 ml static cultures seeded with 1×10⁷ bacteria. To measure thermotolerance, logarithmic cultures were incubated at 53° C. and subsequent viability assessed by plating at 37° C.

Transcriptional start sites were located using RNA extracted from cultures of BCG and the corresponding ΔhspR mutant grown at 37° C., with or without heat shock for 45 min at 45° C., as described by Mangan et al¹⁵. γ[³²P]-labelled primer (PEX1, 5′-CCTCCTGAATATGTAGAG-3′) (SEQ ID NO: 14) was annealed with 40 μg total RNA in reverse transcription buffer, and extension carried out at 42° C. for 60 minutes following addition of 500 μM dNTPs, 40 U of RNAsin (Promega, Southampton, U.K), 5 mM dithiothreitol (DTT), and 200 U of Superscript II reverse transcriptase (Life Technologies, Carlsbad, Calif., USA). Extension products were visualised by separation on a 6% polyacrylamide-urea sequencing gel against a DNA sequence ladder generated using a standard sequencing primer with single strand M13 bacteriophage DNA.

Protein synthesis was monitored in mid-logarithmic phase bacterial cultures (10 ml) resuspended in 1 ml of Middlebrook 7H9 medium containing 10 μCi of [³⁵S]-methionine (specific activity >1000Ci/mol). After incubation for 90 min at 37° C. or 45° C., protein extracts were prepared in SDS-PAGE sample buffer, adjusted to 10,000 cpm/μl, and analysed by SDS-PAGE and autoradiography.

For two-dimensional electrophoresis, protein extracts prepared by bead beating of logarithmic cultures were lyophilised, resuspended in isoelectric focusing (IEF) sample buffer (6M urea, 2 M thiourea, 2% Triton X-100, 1 mM DTT, 4% ampholytes pH 4-6 and 1% ampholytes pH 3-10) and separated by IEF in tube gels and then by SDS-PAGE in a second dimension. For MS-analysis, excised protein spots were reduced, carboxyamidated and digested in situ with trypsin. Samples were centrifuged and analysed by MALDI mass spectrometry performed on a VG TofSpec SE time-of-flight (TOF) mass spectrometer equipped with a delayed extraction ion source (Micromass, Cary, N.C., USA). Spectra were internally calibrated using the matrix ion at m/z 1060.10 and trypsin autolysis peaks at m/z 2163.06 and m/z 2289.15. Monoisotopic masses were assigned and proteins identified by peptide mass fingerprinting using PepSea software (Protana, www.protana.com) and a mass accuracy of 0.1 Da.

### Example 3

### Infection Models

Bone marrow-derived macrophages were cultivated and infected with mycobacteria as previously described⁴³ but using Macrophage-SFM Medium (Life Technologies) supplemented with 10 ng/ml IL-3 (Pharmingen, Franklin Lakes, N.J., USA).

6-8 week old C57BL/6 mice were inoculated with 1×10⁶ bacteria by-tail vein injection. Groups of mice (n=4) were culled and weighed at day 1, and 2, 4, 6, 8, 10 and 14 weeks post-infection. Spleens and lungs were weighed and bacterial load assessed by serial dilution of organ homogenates on 7H11 agar plates. Organs were also fixed in formal saline, embedded in paraffin, and sections of approximately 5 μm were cut and stained with hematoxylin and eosin.

### Example 4

### Immunological Analysis

C57BL/6 mice were infected intravenously with 2×10⁵ CFU BCG or BCG ΔhspR. Animals were culled at day 14 and 35 after infection by intraperitoneal injection of 3 mg pentobarbitone and exanguination via the femoral vessels. Single cell lung and spleen suspensions were obtained by homogenizing tissues through 100 μm cell strainers.

Cells were stained with Quantum red-conjugated (QR) anti-B220 (CD45R), FITC-conjugated anti-CD45RB, cychrome-conjugated (Cy) anti-CD4 and phycoerythrin-conjugated (PE) anti-CD8 and anti-NK1.1 (all from Pharmingen) for 30 minutes on ice and with 2% formaldehyde. To detect intracellular cytokines, 10⁶ cells per ml were incubated with 50 ng/ml PMA, 500 ng/ml ionomycin and 10 mg/ml brefeldin A for 4 hours at 37° C. Cells were then stained for CD8-PE and CD4-Cy as described above and fixed. After permeabilising with 0.5% saponin in PBS containing 1% BSA and 0.1% azide for 10 minutes, FITC-conjugated anti-IFN-γ (XMG1.2, Pharmingen) diluted 1/40 in saponin buffer was added. After 30 minutes all samples were washed with PBS containing 1% BSA and 0.1% sodium azide and analyzed on a Becton Dickinson (Franklin Lakes, N.J., USA) FACSCAN™ flow cytometer collecting data on at least 40,000 lymphocytes.

For ELISPOT assay, sterile filter plates were coated with rat anti-murine IL-4, and IFN-γ antibodies (Pharmingen), washed and blocked with RPMI containing 10% fetal calf serum. Splenocytes were added to the wells at 10⁶ cells/well with 4 doubling dilutions. Cells were cultured for 48 hours with medium alone or 10 μg/ml purified M. tuberculosis Hsp70⁴². The sites of cytokine production were detected using biotin-labelled rat anti-murine IL-4, or IFN-γ monoclonal antibodies (Pharmingen) as previously described⁴⁵.

Statistical analysis. Statistical comparisons were made using Student's t-test and P<0.05 was considered significant.

### Example 5

### Characterisation of Hsp70 Regulation in M. tuberculosis

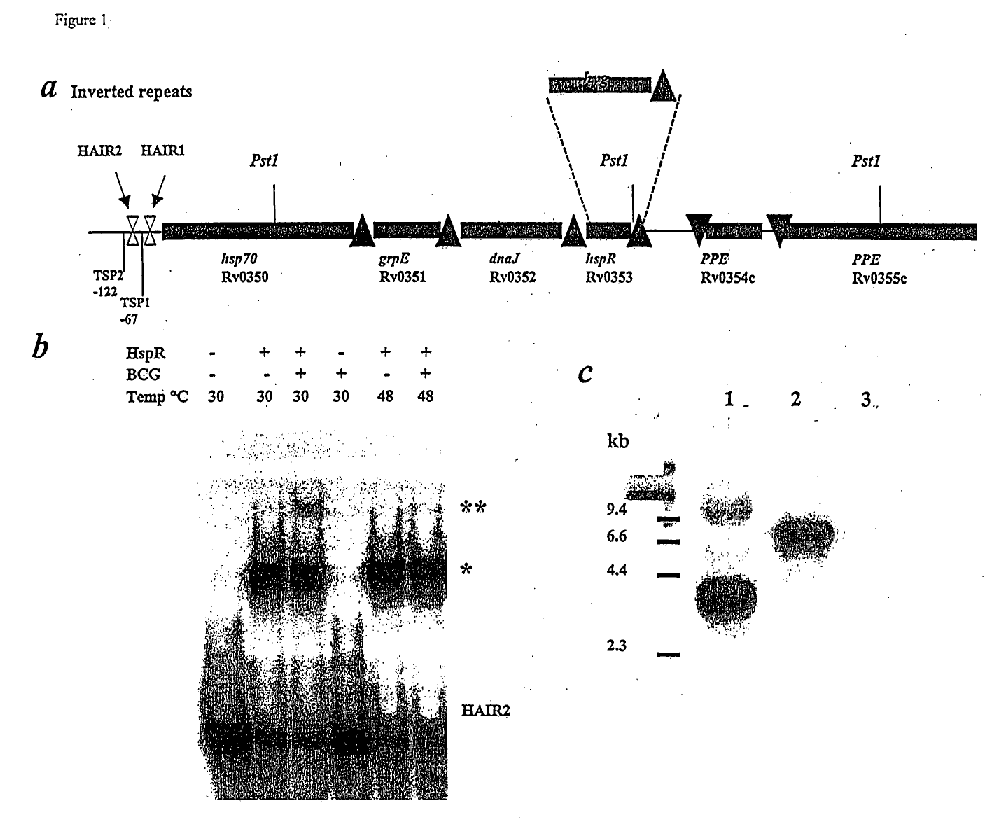

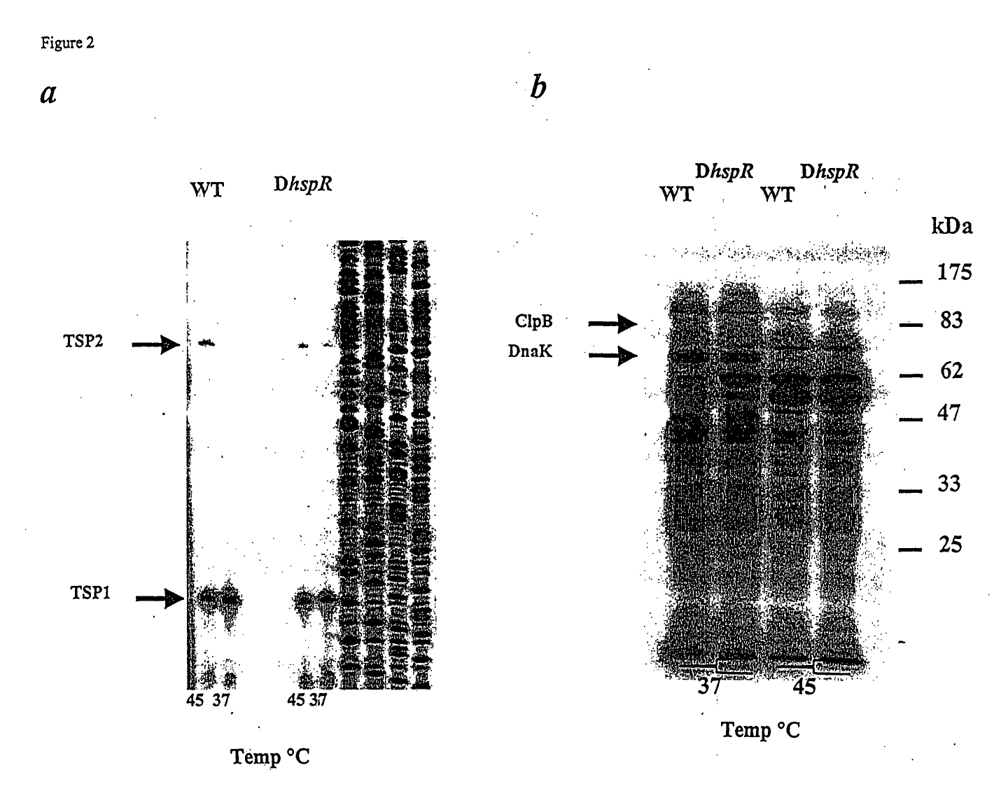

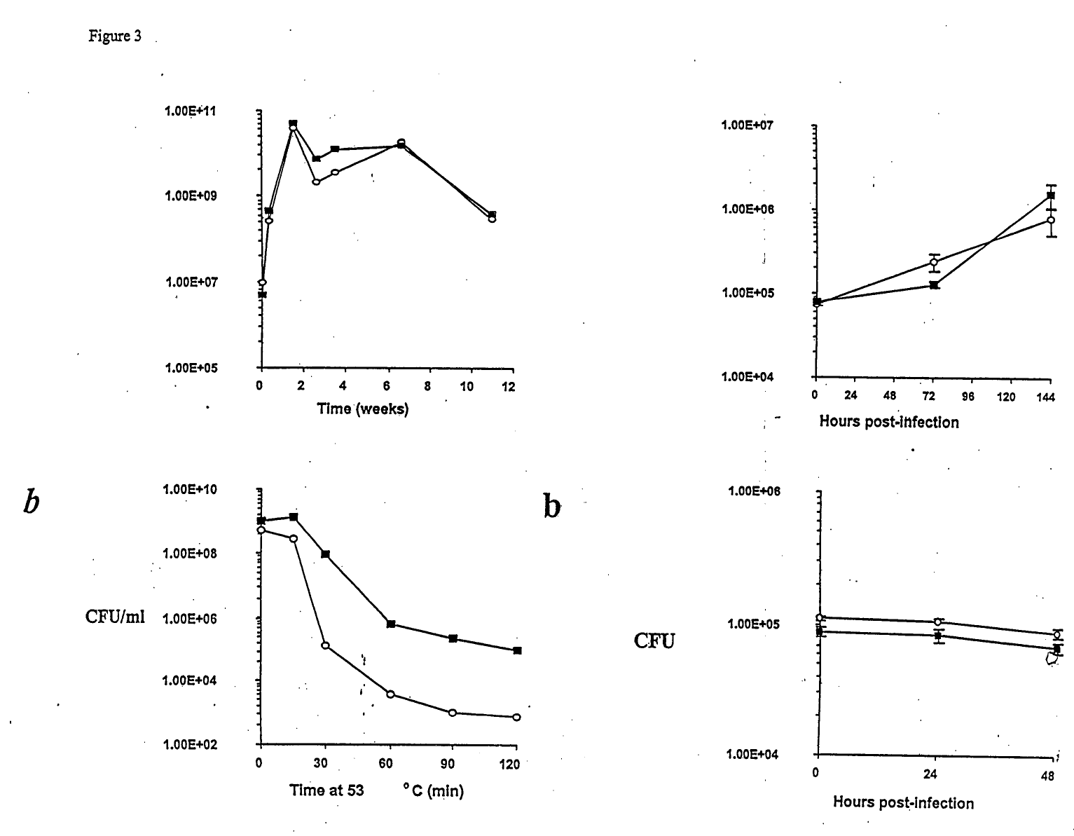

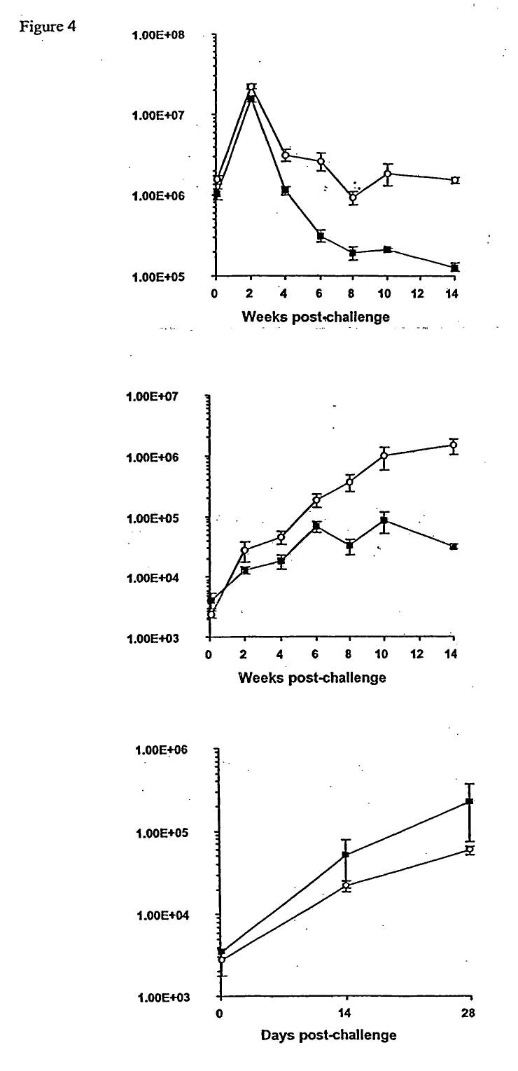

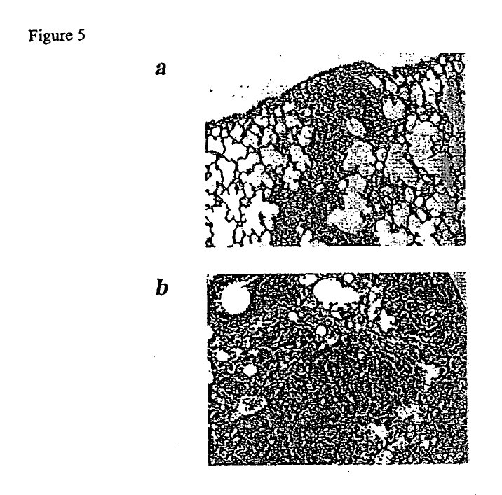

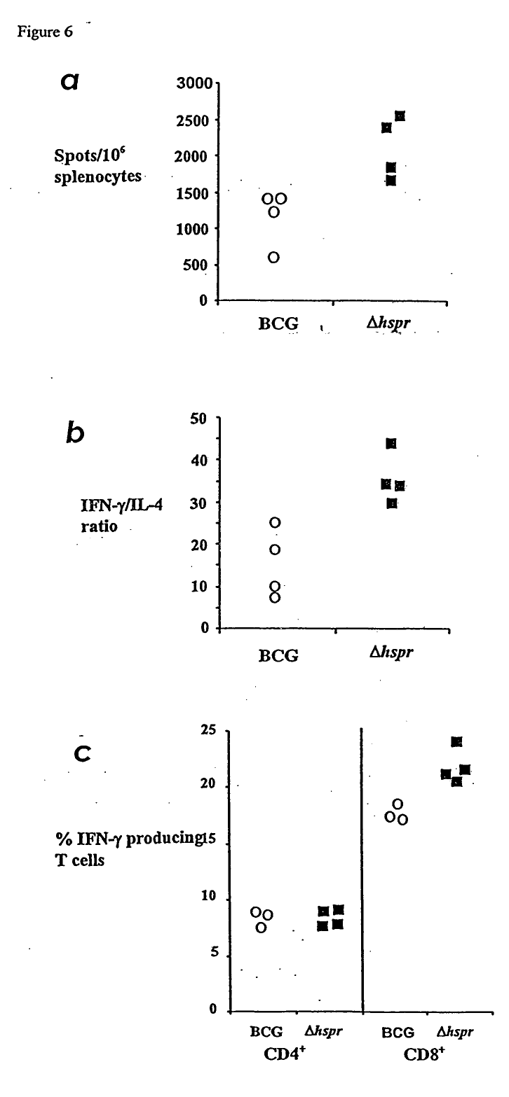

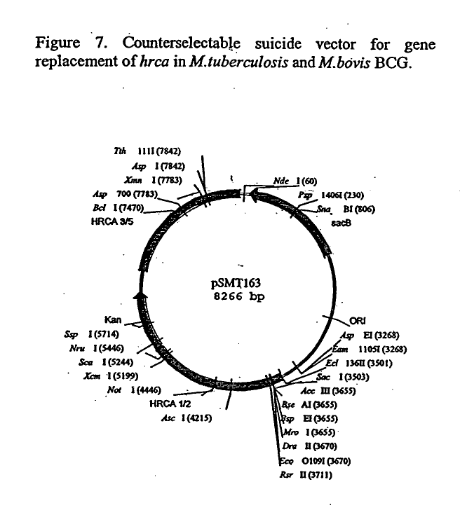

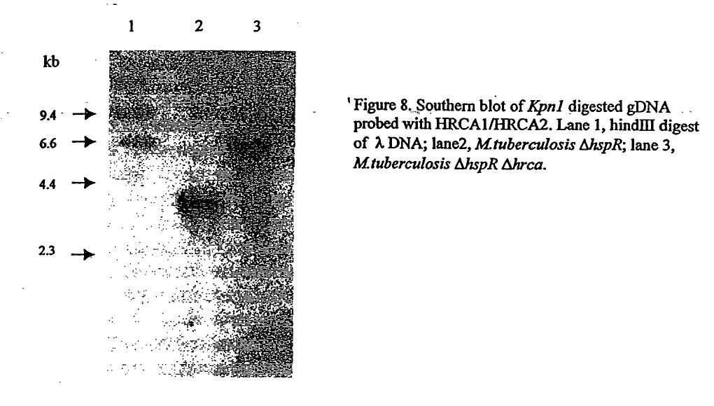

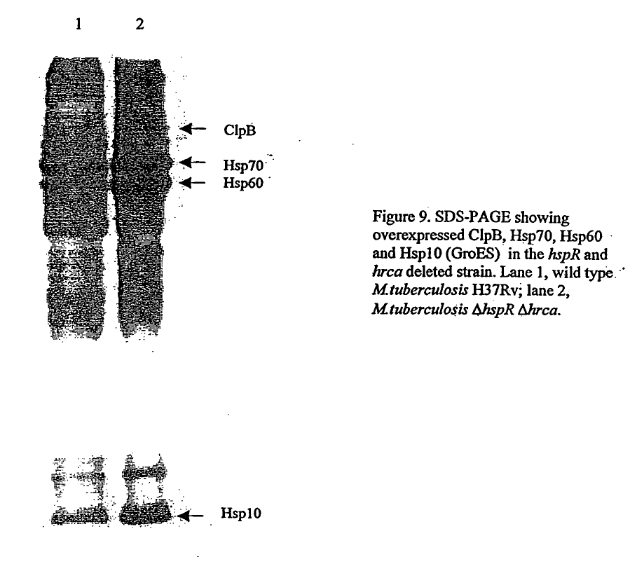

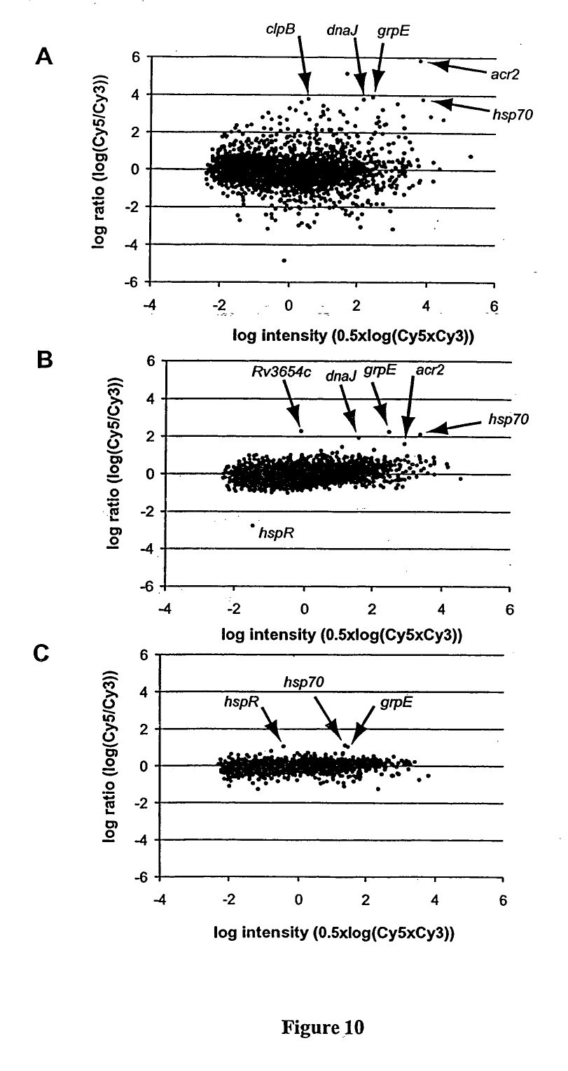

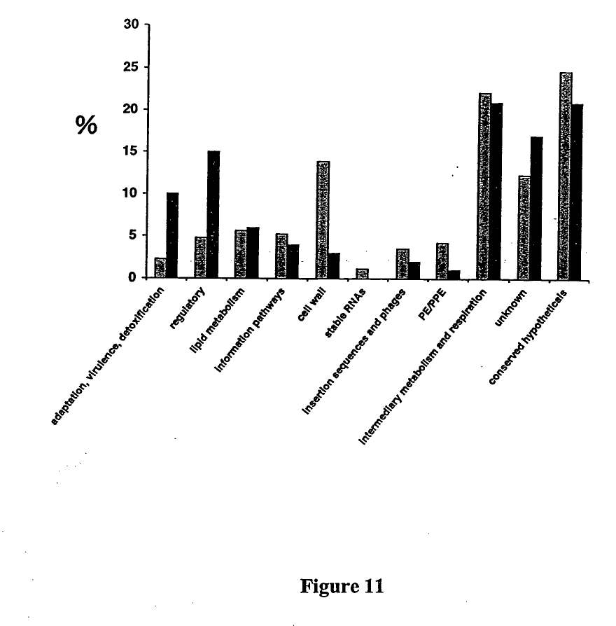

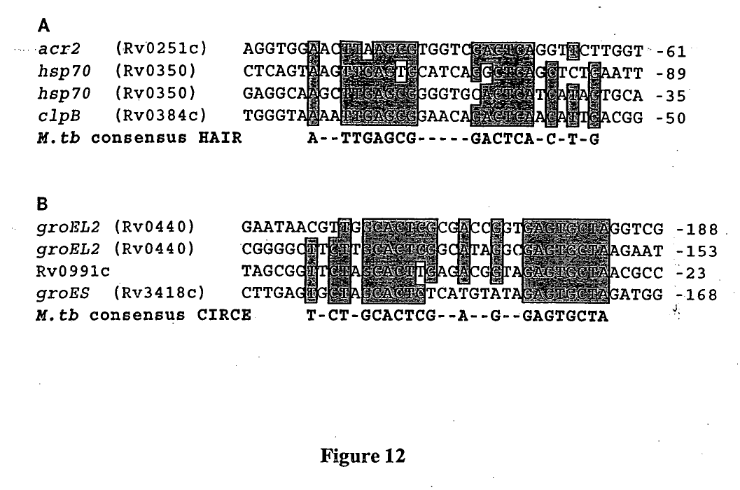

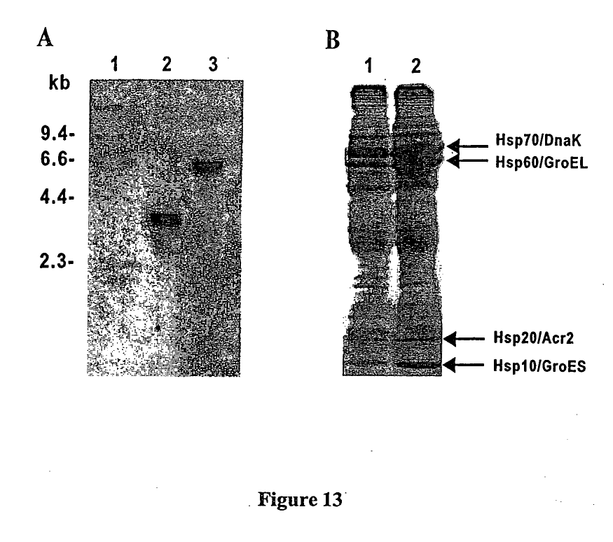

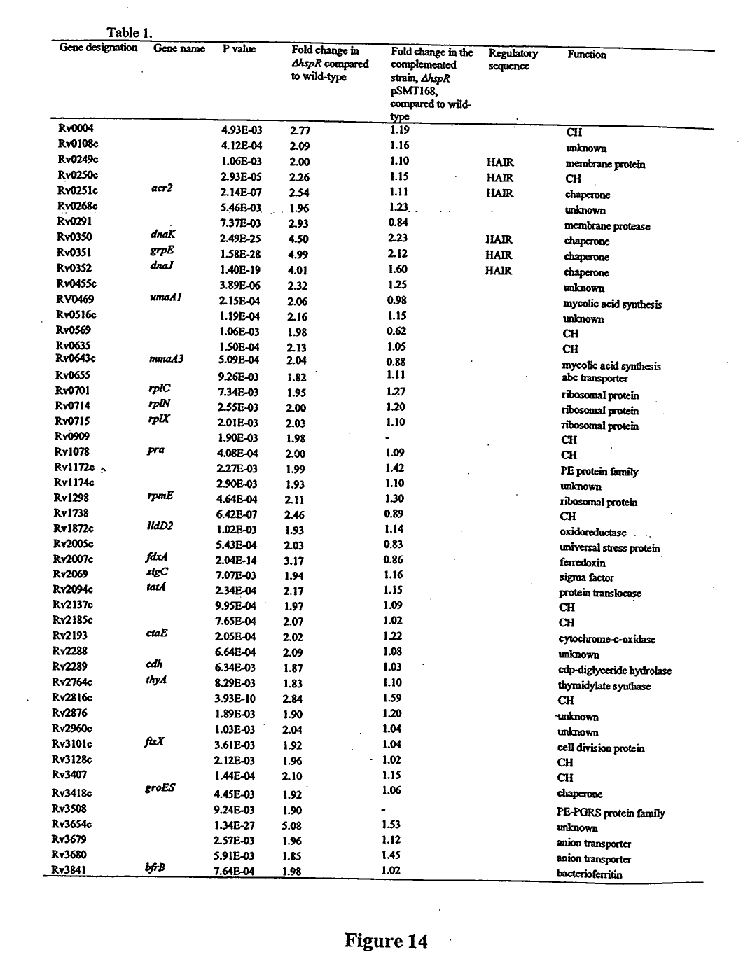

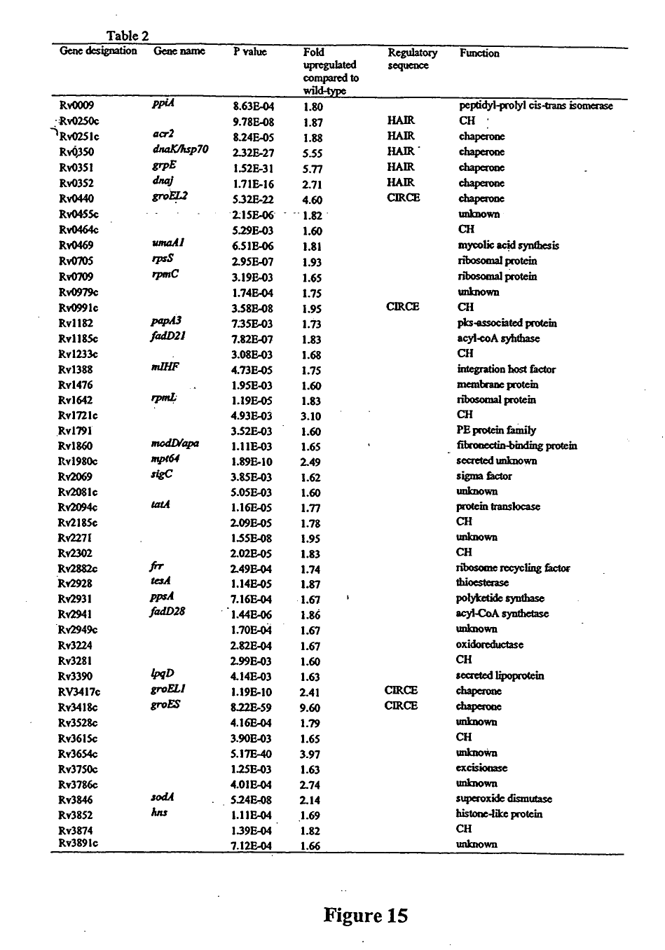

Exposure of M. tuberculosis to increased temperature results in elevated transcription of heat shock genes and expression of the corresponding proteins^{15,16}. The regulatory mechanisms involved have not been characterized. Two general mechanisms for heat shock regulation have been identified in bacteria. Induction of the response in E. coli involves transcriptional activation, with increased levels of an alternative sigma factor, sigma-32, directing RNA polymerase towards genes preceded by a consensus heat shock promoter sequence¹⁷. In contrast, in Bacillus subtilis the heat shock response is regulated by transcriptional repression¹⁸. In unstressed cells, the HrcA repressor blocks transcription by binding to an inverted repeat element upstream of the heat shock genes, with repression being released in response to stress stimuli. Inspection of the genome sequence of M. tuberculosis¹⁹ suggests repression as the probable mechanism of heat shock regulation. Open reading frame Rv2374c encodes a homologue of the HrcA repressor, while Rv0353 encodes a protein similar to HspR, a repressor identified in Hsp70 regulation in Streptomyces²⁰ and in Helicobacter pylori²¹. The M. tuberculosis hspR is the fourth gene in an operon comprising Hsp70, followed by genes encoding GrpE and DnaJ, heat shock proteins that have functional interactions with Hsp70²² (FIG. 1a).

To test whether M. tuberculosis HspR has a function analogous to the Streptomyces homologue, it was expressed as a His-tagged protein and characterized in a gel shift assay (FIG. 1b). HspR bound to a 40 bp oligonucleotide corresponding to a region upstream of M. tuberculosis Hsp70 containing a partial match for the HspR-associated inverted repeat (HAIR) identified in Streptomyces^{20,23}. HspR showed no binding to a control irrelevant oligonucleotide. The effect of heat shock on the HspR-HAIR interaction was tested by carrying out the reaction at 48° C. Heating had no effect on the gel shift pattern. An effect of heat shock was observed, however, when a mycobacterial extract was included in the assay. Reaction of the oligonucleotide with HspR and the cell extract at low temperature, 30° C., produced a second gel shift band (FIG. 1b, lane 3). This second, super-shifted band was absent when binding was carried out at 48° C. (lane 6).

The ability to bind to the upstream regulatory sequence suggests that M. tuberculosis HspR has a function analogous to that of its Streptomyces counterpart²⁰. The presence of the temperature-sensitive super-shifted band is consistent with a model in which HspR and Hsp70 together form the functional repressor, with sequestration of Hsp70 as a result of binding to denatured proteins releasing repression during heat shock²⁴.

### Example 6

### Deletion of the HspR Repressor

Taking advantage of sacB counter-selection²⁵, an allele replacement strategy was used to substitute the hspR gene with a hygromycin resistance cassette in M. tuberculosis and BCG (FIG. 1c).

Expression of the hsp70 operon in wild type M. bovis BCG and the ΔhspR mutant was compared by mapping of transcriptional start points (FIG. 2a) In the wild type strain grown at 37° C., a single start site (TSP1) was identified at position −67 in relation to the initiation codon. A second transcript initiating at position −122 (TSP2) was observed in cells that had been heat shocked. In the mutant, transcription occurred from both sites even in the absence of heat shock. TSP1 and TSP2 are located 5 bases and 6 bases upstream of HAIR1 and HAIR2 respectively. While transcription from both sites is therefore likely to be influenced by HspR, the mapping results demonstrate that this effect is more pronounced in the case of the TSP2 transcript.

Next the pattern of protein expression in the ΔhspR mutants was analyzed. The response was the same in M. tuberculosis and BCG. The SDS-PAGE profiles of newly synthesised proteins labeled with [³⁵S]-methionine at −37° C. and 45° C. (FIG. 2b) showed that Hsp70 was induced in the wild type strains at the elevated temperature. In the mutants, however, this band was equally prominent in the control 37° C. cultures. Other less marked differences included constitutive overexpression of bands at 90 kDa and 45 kDa in the ΔhspR mutants, again corresponding to changes induced by heat shock in the wild type. The changes in protein profile were further characterized by two-dimensional gel electrophoresis. Three protein spots were upregulated in the mutant and were identified by peptide mass fingerprinting as Hsp70, ClpB, and GrpE. DnaJ, the third heat shock protein in the hsp70 operon, has a relatively basic isoelectric point (predicted pI 8.05) and was not resolved.

Results generated using the deletion mutants were again consistent with the model in which HspR acts as a repressor of the hsp70 operon. To confirm that the effects were due solely to the loss of hspR, the cloned gene was reintroduced using mycobacterial expression vectors. These experiments were unsuccessful. Plasmids constitutively expressing HspR could not be maintained in mycobacteria. Although it was possible to introduce the hspR gene into M. tuberculosis using the inducible acetamidase promoter⁶, induction of HspR expression resulted in cessation of bacterial growth. Thus, while deletion of hspR is well-tolerated by M. tuberculosis, it seems that inappropriately regulated expression has a profound detrimental effect on bacterial viability. The location of the hspR gene at the end of the hsp70 operon, and its reverse orientation with respect to the adjacent downstream PPE gene (Rv0354c) (FIG. 1a), suggests that polar effects are unlikely to cause the mutant phenotype, but failure to complement the mutation leaves the possibility that the mutant phenotype is due to an unrelated mutation.

### Example 7

### Phenotype of the ΔhspR Mutant In Vitro and During Infection

The M. tuberculosis and BCG mutants were slightly impaired for in vitro growth. Colonies on solid media were marginally smaller than wild type after 2 weeks growth and the doubling time of the M. tuberculosis mutant (20.0 hours±0.2 (SE)) was greater than wild type (19.3 hours±0.1 (SE)) in liquid medium. Survival in stationary phase cultures was indistinguishable from wild type controls (FIG. 3a). A significant difference was observed in thermotolerance, with survival of the ΔhspR mutant at 53° C. enhanced in comparison to that of the parent strain (FIG. 3b).

The M. tuberculosis ΔhspR mutant was compared to the parent strain in its ability to survive in murine bone marrow macrophages. Both mutant and wild type strains were able to replicate in quiescent macrophages that had been cultured in the absence of deliberate exogenous activation signals. There was no significant difference between the rates of replication (FIG. 3c). In activated macrophages a state of apparent bacteriostasis was achieved, again with no difference in CFU counts between mutant and wild type (FIG. 3d).

Next, the ability of the ΔhspR mutant to cause progressive infection in C57BL/6 mice was examined. In this model, the bacteria were seeded in multiple organs and underwent an active expansion during an initial acute phase of infection. Induction of a cell-mediated immune response after a few weeks resulted in partial control of the infection, initiating a chronic phase of relatively constant bacterial load. Mice survive in the chronic phase for many months, ultimately expiring as a result of progressive damage to the lungs²⁷. In the spleen, growth of the mutant was identical to wild type M. tuberculosis during the initial acute phase of infection, but there was a ten-fold reduction in CFU in the chronic phase (P<0.001 at 14 weeks) (FIG. 4a). This reduction in bacterial load, which was observed in repeat experiments, matches that achieved by prior immunization of mice with BCG in this model. A significant difference was also observed in the lungs of the same animals, with a 1-2 log reduction in bacterial load in the mice infected with M. tuberculosis ΔhspR at 14-weeks (P=0.016)(FIG. 4b). For accurate assessment of the low numbers of bacteria during the initial phase of infection in the lung, data were combined from three independent experiments. There was no significant difference between mutant and wild type during this acute phase; in fact, in two of the three experiments, growth of the mutant-in lung tissues surpassed that of the wild type over the first four weeks (FIG. 4c).

There was no evidence to suggest that the reduction in bacterial load was associated with increased immune-mediated pathology. The mean weight of animals at 10 and 14 weeks was slightly higher in the ΔhspR group (25.35 g) compared to wild type (23.92 g)(P=0.058). Histological examination of lungs from ΔhspR mice revealed small, isolated macroscopic lesions consisting mainly of macrophages with scattered lymphocytes and polymorphonuclear leucocytes (FIG. 5a). The majority of the lung retained a healthy morphology with thin or only slightly thickened alveolar septa and patent airways. Lungs from the wild type infections contained many more lesions, which were considerably larger, consisting sheets of macrophages with tight wedges of lymphocytes. Alveolar septa were thickened throughout the lung and there was some coalescence of granulomas, leading to a substantial reduction in patency of airways (FIG. 5b).

### Example 8

### Immune Response to the ΔhspR Mutant

To test the hypothesis that reduced survival of M. tuberculosis ΔhspR during chronic infection could be due to a heightened immune response, the effect of Hsp70 overexpression on immunogenicity was investigated. Immune responses of mice infected intravenously with wild-type or BCG ΔhspR were analyzed. As with M. tuberculosis, the wild-type and mutant strains survived similarly during acute infection, with no significant difference in CFUs at day 14. ELISPOT analysis of Hsp70-stimulated splenocytes at day 35 revealed a two-fold increase in the number of IFN-γ producing cells from mice infected with BCG ΔhspR compared to wild type (P=0.02) (FIG. 6a). The ratio of IFN-γ:IL4 producing Hsp70-specific splenocytes was also increased two-fold following BCG ΔhspR infection (P=0.02) (FIG. 6b). Analysis of cell populations by flow cytometry did not reveal any significant difference in the number of lymphocytes, CD4⁺ and CD8⁺ T cells, NK cells and B cells in the lung and spleen 14 or 35 days after infection, nor was there a significant difference in expression of the activation marker CD45RB on T cells. However, in the spleen the number of CD8⁺ (but not CD4+) T cells secreting IFN-γ was significantly higher in the BCG ΔhspR infected group (P=0.009) (FIG. 6c). This increase in CD8⁺ IFN-γ producing cells was larger than could be explained solely by the increase in Hsp70-specific IFN-γ secreting cells observed by ELISPOT.

### Example 9

### Dissection of the Heat Shock Response to M. tuberculosis using Mutants and Microarrays

Experimental Procedures

Bacterial Strains and Growth Conditions

All DNA vector construction was performed in Escherichia coli DH5a. E. coli were grown at 37° C. in Luria Bertani broth and agar containing 150 μg/ml hygromycin or 50 μg/ml kanamycin where appropriate. M. tuberculosis H37Rv, ΔhspR and ΔhspR ΔhrcA were grown at 37° C. in Middlebrook 7H9 broth (Difco) containing 10% albumin dextrose catalase (ADC) enrichment or on Middlebrook 7H11 agar medium (Difco) containing 10% oleic acid, dextrose, albumin, catalase (OADC) enrichment. Hygromycin at 50 μg/ml and kanamycin at 15 μg/ml were added where appropriate. 2% sucrose was added to media for counterselection of sacB. Heat shock was performed by splitting 20 ml broth cultures at late log phase into two universal tubes and placing one tube at 37° C. and the other at 45° C. for 30 minutes.

Deletion of hspR, hrcA in M. tuberculosis

The gene replacement of hspR with the hygromycin B phosphotransferase gene (hyg) from Streptomyces hygroscopicus has been previously described.⁶⁸ The sequential deletion of hrcA to generate a double hspR hrcA mutant strain was achieved using a similar suicide delivery strategy but replacing the target gene, hrcA, with the kanamycin resistance gene (aph) from Tn903. Briefly, 1.5 kb regions of DNA up and downstream of hrcA were cloned around the aph gene in the mycobacterial suicide plasmid pSMT99 to make pSMT163. This plasmid cannot replicate in mycobacteria and carries sacB for counterselection against single crossover and illegitimate integration of the plasmid. 1 μg of plasmid was irradiated with 100 mj/cm² UV⁵⁸ and electroporated into M. tuberculosis or M. tuberculosis ΔhspR.⁷² Following overnight recovery of the cells in 7H9/ADC, gene replacement transformants were directly selected on 7H11/OADC containing hygromycin, kanamycin and sucrose. Deletion of hrcA was confirmed by Southern blotting of Kpn1 digested genomic DNA using the 1.5 kb upstream hrcA fragment as hybridisation probe.

Complementation of M. tuberculosis ΔhspR

pKinta is a Co1E1 based E. coli plasmid which carries the aph kanamycin resistance gene and the int gene and attP site from the L5 mycobacteriophage.⁶⁹ This plasmid integrates into the chromosome in single copy by site-specific recombination at the attB site. The Hsp70 operon promoter containing the two HAIR-regulated promoter regions⁶⁸ was amplified by PCR using the primers Hsp701 (tcggtcaagctggcggactga) (SEQ ID NO: 14) and Hsp702 (agccatggtgaatcctcctg) (SEQ ID NO: 15) and cloned into the Sac1 site of pKinta. The hspR ORF was then amplified and cloned downstream of the hsp70 promoter so as to transcriptionally fuse the ORF with its own promoter albeit without the intervening hsp70, grpE and dnaJ sequence. The resultant plasmid, pSMT168, was introduced to M. tuberculosis ΔhspR by electroporation.

RNA Extraction and cDNA Labeling

10 ml of broth culture in late log phase was added directly to 40 ml of GTC solution containing 5 M guanidinium thiocyanate, 0.5% sodium N-lauryl sarcosine, 0.1 M β-mercaptoethanol, 0.5% Tween 80. The bacteria were pelleted by centrifugation and resuspended in 1.2 ml Trizol (Life Technologies). The phases were separated by the addition of 0.6 ml chloroform, mixing and centrifugation. The aqueous phase was reextracted with chloroform and the RNA precipitated with isopropanol, washed in 70% ethanol and dissolved in Rnase-free water. The RNA was treated with amplification grade Dnase I (Life Technologies) and cleaned up by RNeasy purification (Qiagen).

cDNA was labelled by incorporation of Cy3 or Cy5 dCTP (Amersham) during reverse transcription of RNA. 2-10 μg RNA was mixed with 3 μg of random hexamer oligonucleotides in 11 μl water, heated to 95° C. and snap cooled. In a total volume of 25 μl the labelling reaction was initiated by the addition of 5 μL First Strand Buffer, 25 mM DTT, 1 mM each DATP, dGTP, dTTP, 0.4 mM dCTP, 2 nmol Cy3- or Cy5-dCTP and 500 U Superscript II reverse transcriptase (Life Technologies). The reaction was incubated in the dark at 25° C. for 10 min and then at 42° C. for 90 min. The relevant pairs of Cy3 (wild-type H37Rv) and Cy5 (mutant strain or heat shocked cells) labelled cDNA were mixed and purified using a Qiagen MinElute kit, eluting in water.

Microarrays and Hybridisations

Whole genome microarrays were constructed by robotic spotting onto poly-lysine coated glass microscope slides (MicroGrid II, BioRobotics, UK) of PCR amplicons derived from each of the 3924 ORF's of the sequenced strain of M. tuberculosis H37RV. Primer pairs for each ORF were designed with Primer 3 software and selected by BLAST analysis to have minimal cross-homology with all other ORF's. All procedures used including post-processing of deposited arrays were as described by others.⁷³[Wilson M, 2001 #38].

The microarray was incubated in prehybridisation solution (3.5×SSC, 0.1% SDS and 10 mg/ml BSA) at 65° C. for 20 min. The slide was rinsed in water for 1 min and propan-2-ol for 1 min before drying by centrifugation at 1500 rpm for 5 min.

The purified Cy3/Cy5 labelled cDNA was adjusted to 16 μl in 4×SSC and 0.3% SDS. This hybridisation solution was heated to 95° C. for 2 min, briefly centrifuged and applied to the array under a cover slip. The slide was sealed in a humid hybridisation cassette and incubated at 65° C. in the dark for 16-20 h. The slide was washed for 2 min at 65° C. in 1×SSC/0.05% SDS, for 2 min in 0.06×SSC at room temperature and then dried by centrifugation. The hybridised microarrays were scanned with an Affymetrix 428 scanner. The scanned images were analysed with ImaGene4.1 and the median spot intensities calculated.

Data Processing and Statistical Analysis

For each strain or condition 3 or 4 independent RNA preparations were analysed. Background values were subtracted from signal values. In cases where this resulted in negative values, a small positive constant was assigned to prevent numerical problems when forming ratios or taking logarithms. All values were log₂ transformed for further analysis.

Significance values were calculated for each ORF in the mutant:wild-type comparisons through an ANOVA analysis. Each of the three data sets (wild-type v ΔhspR; wild-type v ΔhspR pSMT168; wild-type v ΔhspRhrcA) forms a balanced factorial design. Three main effects were taken into account: the array effect A for each array, the gene effect G for each gene, and the variety effect V for the two varieties, mutant and control. In addition, the three pairwise interactions between the main effects, that is, interactions A:V, A:G, and V:G have been accounted for. The resulting residuals stem from the A:V:G interaction of all three main effects which were used to estimate the standard error. One problem is that the residual or error variance is much higher for low expression values, which is not unexpected considering the higher uncertainty in these values. Hence, using one standard error estimate for all genes does not seem appropriate. Instead, we resampled from the residuals, redistributed them over the expected response values and fitted new models to these bootstrap replicates. The multitude of models allowed us to calculate confidence intervals for the estimates of the effects. The final value is based on the difference in estimated V:G effects, which represents the influence of variety, that is, mutant or control, on gene expression. Confidence intervals for these effects are calculated through the resampling procedure as above. Final p-values are obtained from confidence intervals by Bonferroni correction for multiple testing, that is, all raw p-values are multiplied by the number of genes resulting in the final adjusted p-values.

Results

Overview of the M. tuberculosis Heat Shock Response

Previous reports have described the induction of heat shock proteins in cultures of M. tuberculosis exposed to temperatures ranging from 37-48° C. for varying lengths of time, and demonstrated transcriptional regulation of selected heat shock genes.^{65,76} These studies demonstrate a complex response, which varies with both temperature and time of exposure. To obtain an overview of the heat shock response, we used whole genome microarray analysis to generate a transcriptomic snap-shot of the changes induced by incubation at 45° C. for 30 minutes; conditions previously demonstrated to result in high level expression particularly of the Hsp70 regulon. This is displayed in the scatter plot (FIG. 10A), which shows the global nature of the transcriptional changes induced by heat shock; the expression ratio of many genes lying away from the zero line demonstrating altered expression. A list of the 100 most highly induced ORFs is provided as supplementary data http://www.cmmi.ic.ac.uk/hsarray.html. The functional distribution of the induced genes varied from that found across the genome, with a bias towards heat induction of adaptation/detoxification and regulatory genes, and away from cell wall associated genes (FIG. 11). The induced genes included all the known members of the HspR regulon, as well as the groEL and groES genes and other previously identified heat shock inducible genes including those encoding the alternative sigma factors σB, σH and σE.^{52,60} This set of heat-inducible genes included five of the nine genes preceded by a σE consensus promoter sequence⁶¹ and all seven genes identified by Raman et al as containing σH consensus promoter regions.⁶⁶ This is consistent with identification of these sigma factors as both heat-inducible genes and regulators of the heat shock response. To characterize regulation of genes encoding the major heat shock proteins, we next extended the microarray approach to analysis of mutant strains of M. tuberculosis from which predicted transcriptional repressors had been deleted.

The HspR Regulon

By examining the gene expression profile at 37° C. of an M. tuberculosis strain lacking the transcriptional repressor HspR (ΔhspR), we aimed to isolate any de-repressed genes and identify the subset of heat inducible genes directly under HspR control. In contrast to the heat shocked bacteria, transcription of the majority of genes was unaltered in the mutant strain, but there were several obvious upregulated genes (FIG. 10B). ANOVA analysis also revealed the less obvious upregulated genes, exposing a set of 49 upregulated ORFs (p<0.01) in the mutant strain, including the members of the Hsp70 operon (dnaK, grpE and dnaJ) (FIG. 14, Table 1).

We searched the genome for sequences that resembled the HspR binding site, HAIR (HspR Associated Inverted Repeat) CTTGAGT-N-7-ACTCAAG (SEQ ID NO:3)⁵³, and compared the locations of potential sites to the gene expression analysis of both heat shocked M. tuberculosis and M. tuberculosis ΔhspR. In addition to the HAIR sequences already identified upstream of the Hsp70 operon and clpB⁶⁸, a HAIR-like domain was present 71 bp upstream of the start codon of Rv0251c (FIG. 12A). This gene bears similarity to the α-crystallin (acr)/14 kD antigen of M. tuberculosis (41% identity over 98 amino acids), so we have termed it acr2. It appears to be at the head of an operon preceding Rv0250c and Rv0249c as these are also upregulated in the mutant (FIG. 14, Table 1). The genomic organization is also consistent with Rv0248c and Rv0247c (predicted to encode an oxidoreductase) being members of the operon. Neither of these genes was detected as significantly upregulated in the ΔhspR mutant by ANOVA analysis. There were no other HAIR-like sequences associated with any of the other up-regulated genes in the ΔhspR strain.

As expected the Hsp70 operon genes along with acr2 and Rv0250c were upregulated in response to heat shock. Under the conditions used in this study, acr2 was the most heat inducible gene in the genome (FIG. 10A). Other ΔhspR-regulated ORFs demonstrated to be induced under heat shock were Rv3654c, bfrB and groES. Rv3654c encodes an 8 kD protein of unknown function and bfrB encodes a bacterioferritin involved in iron aquisition; neither gene has an identifiable HAIR like sequence in its vicinity and both are therefore concluded to be under some indirect control by HspR. Most interesting, is the inclusion of the chaperone gene groES as our previous studies had not indicated that this gene was controlled by HspR. Indeed the level of induction is considerably less than that of the Hsp70 or Acr2 operons. The HspR associated control over groES expression may be indirect as there is no HAIR sequence in the promoter region, however there is a weak HAIR-like sequence situated 24 bases downstream of the groES initiation codon. The remaining non-heat-induced genes upregulated in the ΔhspR mutant presumably reflect adaptive responses triggered by constitutive overexpression of the genes normally controlled by HspR. Notable members of this group included genes encoding the alternative sigma factor σC, the sec-independent protein translocase, TatA, and also four ribosomal proteins. Indeed, there was a general trend among nearly all the ribosomal protein genes to be upregulated in the ΔhspR mutant.

We had previously described unsuccessful attempts to complement the M. tuberculosis ΔhspR strain.⁶⁸ Reintroduction of the gene with a constitutive promoter or even gently induced expression from the acetamidase promoter⁶⁴ rendered the bacteria non-viable. These findings suggest that expression of reintroduced hspR would have to be appropriately regulated so as to closely match wild-type expression dynamics. To achieve this, the hspR gene was cloned under the control of the natural promoter of the hsp70 operon, which includes two HAIR sequences. A single copy of this construct was inserted at the attB phage integration site in the chromosome of M. tuberculosis ΔhspR. In contrast to previous attempts at complementation, this strain was fully viable. Whole-genome expression profiling of the complemented mutant showed a pattern largely similar to the original wild-type strain (FIG. 10C). The reintroduced hspR gene was approximately 2-fold over-expressed demonstrating that the complementing construct did not express hspR identically to wild-type, perhaps reflecting some stoichiometric relationship between hspR expression and the number of HAIR sites. However, all the genes overexpressed in the ΔhspR strain showed a complete or substantial reduction of overexpression in the complemented strain (FIG. 14, Table 1). This demonstrates that the altered transcriptome of the mutant was specifically due to the absence of hspR and not to polar effects on neighboring genes or to an inadvertently selected mutation.

The HrcA Regulon

ORF Rv2374c in the M. tuberculosis genome shares sequence homology with the family of heat shock repressors related to the hrcA gene of B. subtilis. To test whether this ORF is similarly involved in heat shock regulation in M. tuberculosis we undertook a deletion strategy analogous to that used to generate the ΔhspR mutant, replacing hrcA with a kanamycin resistance gene. We were unable to generate ΔhrcA mutants in wild-type M. tuberculosis, yet were successful at introducing the mutation into M. tuberculosis ΔhspR (FIG. 13A). SDS-PAGE analysis of the total protein profile of the double knock out M. tuberculosis ΔhspRΔhrcA demonstrated constitutive overexpression of proteins consistent in size with Hsp70, Hsp60 (GroEL) and GroES, as well as an additional band at approximately 20 kD (FIG. 13B).

Whole-genome expression profiling of M. tuberculosis ΔhspRΔhrcA at 37° C. revealed enhanced expression of a set of 48 ORFs (p<0.01) (FIG. 15, Table 2). Twelve ORFs upregulated in the single ΔhspR mutant were also upregulated in the ΔhspRΔhrcA strain. These included members of the Hsp70 and Acr2 operons as well as sigC, tatA and groES. The upregulation of groES was much greater in the ΔhspRΔhrcA mutant than in the ΔhspR strain (9.60 and 1.96 fold respectively). This indicated that although transcription of groES can be induced by an HspR-associated mechanism, the predominant mode of transcriptional control is through the HrcA repressor. HrcA also seemed the likely mechanism of control for the two M. tuberculosis groEL genes as these were both strongly upregulated in the ΔhspRΔhrcA strain. We searched the genome for the HrcA binding site, CIRCE TTAGCACTC-N-9-GAGTGCTAA (SEQ ID NO: 16)⁵⁶ and, as for HspR, compared the putative CIRCE locations with both the heat shock expression data and the double mutant transcriptional profile. groEL2 is preceded by two CIRCE-like elements and groES/groEL1 by one (FIG. 12B). This confirmed the hypothesis that HrcA acts as the main regulator for the GroE/Hsp60 heat shock protein family.

A CIRCE-like sequence was also identified 28 bp upstream of the initiation codon of Rv0991c (FIG. 12B). This ORF is predicted to encode an 11.5 kD conserved hypothetical protein and was significantly upregulated in the ΔhspRΔhrcA mutant (FIG. 15, Table 2). Both Rv0991c and the immediately adjacent downstream gene Rv0990c were upregulated after heat shock for 30 min at 45° C. in the wild-type. Although no significant change was detected in transcription of Rv0990c in the mutant strain, this suggests that the two genes may be coregulated. None of the remaining ΔhspRΔhrcA upregulated genes were associated with CIRCE-like elements nor were they induced under heat shock in the wild-type. Similarly to the single ΔhspR mutant there was a trend for ORFs encoding ribosomal proteins to be upregulated, but in addition the gene encoding ribsome recycling factor, frr, was also significantly upregulated.

It should be understood, of course, that the foregoing relates only to preferred embodiments of the present invention and that numerous modifications or alterations may be made therein without departing from the spirit and the scope of the invention.

### REFERENCES

1. Lindquist, S. & Craig, E.A. The heat-shock proteins. Annu Rev Genet 22, 631-677 (1988).

2. Hartl, F. U. Molecular chaperones in cellular protein folding. Nature 381, 571-579 (1996).

3. Buchmeier, N. A. & Heffron, F. Induction of Salmonella stress proteins upon infection of macrophages. Science 248, 730-732 (1990).

4. Lee, B. Y. & Horwitz, M. A. Identification of macrophage and stress-induced proteins of Mycobacterium tuberculosis. J Clin Invest 96, 245-249′-(1995).

5. Qoronfleh, M. W., Bortner, C. A., Schwartzberg, P. & Wilkinson, B. J. Enhanced levels of Staphylococcus aureus stress protein GroEL and DnaK homologs early in infection of human epithelial cells. Infect Immun 66, 3024-3027 (1998).

6. Cohen, I. R. & Young, D. B. Autoimmunity, microbial immunity and the immunological homunculus. Immunol Today 12, 105-110 (1991).

7. Young, D., Lathigra, R., Hendrix, R., Sweetser, D. & Young, R. A. Stress proteins are immune targets in leprosy and tuberculosis. Proc Natl. Acad Sci USA 85, 4267-4270 (1988).

8. Cho, B. K. et al. A proposed mechanism for the induction of cytotoxic T lymphocyte production by heat shock fusion proteins. Immunity 12, 263-272 (2000).

9. Suto, R. & Srivastava, P. K. A mechanism for the specific immunogenicity of heat shock protein-chaperoned peptides. Science 269, 1585-1588 (1995).

10. Arnold-Schild, D. et al. Cutting edge: receptor-mediated endocytosis of heat shock proteins by professional antigen-presenting cells. J Immunol 162, 3757-3760 (1999).

11. Asea, A. et al. HSP70 stimulates cytokine production through a CD14-dependant pathway, demonstrating its dual role as a chaperone and cytokine. Nat Med 6, 435-442 (2000).

12. Castellino, F. et al. Receptor-mediated uptake of Antigen/Heat shock protein complexes results in major histocompatibility complex class I antigen presentation via two distinct processing pathways. J Exp Med 191, 1957-1964 (2000).

13. Srivastava, P. K., Menoret, A., Basu, S., Binder, R. J. & McQuade, K. L. Heat shock proteins come of age: primitive functions acquire new roles in an adaptive world. Immunity 8, 657-665 (1998).

14. Lowrie, D. B. et al. Therapy of tuberculosis in mice by DNA vaccination. Nature 400, 269-271 (1999).

15. Mangan, J. A., Sole, K. M., Mitchison, D. A. & Butcher, P. D. An effective method of RNA extraction from bacteria refractory to disruption, including mycobacteria. Nucleic Acids Res 25, 675-676 (1997).

16. Young, D. B. & Garbe, T. R. Heat shock proteins and antigens of Mycobacterium tuberculosis. Infect Immun 59, 3086-3093 (1991).

17. Grossman, A. D., Erickson, J. W. & Gross, C. A. The htpR gene product of E. coli is a sigma factor for heat-shock promoters. Cell 38, 383-390 (1984).

18. Hecker, M., Schumann, W. & Volker, U. Heat-shock and general stress response in Bacillus subtilis. Mol Microbiol 19, 417-428 (1996).

19. Cole, S. T. et al. Deciphering the biology of Mycobacterium tuberculosis from the complete genome sequence. Nature 393, 537-544 (1998).

20. Bucca, G., Hindle, Z. & Smith, C. P. Regulation of the dnaK operon of Streptomyces coelicolor A3(2) is governed by HspR, an autoregulatory repressor protein. J Bacteriol 179, 5999-6004 (1997).

21. Spohn, G. & Scarlato, V. The autoregulatory HspR repressor protein governs chaperone gene transcription in Helicobacter pylori. Mol Microbiol 34, 663-674 (1999).

22. Liberek, K., Marszalek, J., Ang, D., Georgopoulos, C. & Zylicz, M. Escherichia coli DnaJ and GrpE heat shock proteins jointly stimulate ATPase activity of DnaK. Proc Natl Acad Sci U S A 88, 2874-2878 (1991).

23. Grandvalet, C., de Crecy-Lagard, V. & Mazodier, P. The ClpB ATPase of Streptomyces albus G belongs to the HspR heat shock regulon. Mol Microbiol 31, 521-532 (1999).

24. Bucca, G., Brassington, A. M., Schonfeld, H. J. & Smith, C. P. The HspR regulon of streptomyces coelicolor: a role for the DnaK chaperone as a transcriptional co-repressordagger. Mol Microbiol 38, 1093-1103. (2000).

25. Pelicic, V., Reyrat, J. M. & Gicquel, B; Expression of the Bacillus subtilis sacB gene confers sucrose sensitivity on mycobacteria. J Bacteriol 178, 1197-1199 (1996).

26. Parish, T., Mahenthiralingam, E., Draper, P., Davis, E. O. & Colston, M. J. Regulation of the inducible acetamidase gene of Mycobacterium smegmatis. Microbiology 143, 2267-2276 (1997).

27. Rhoades, E. R., Frank, A. A. & Orme, I. M. Progression of chronic pulmonary tuberculosis in mice aerogenically infected with virulent Mycobacterium tuberculosis. Tuber Lung Dis 78, 57-66 (1997).

28. Motohashi, K., Watanabe, Y., Yohda, M. & Yoshida, M. Heat-inactivated proteins are rescued by the DnaK.J-GrpE set and ClpB chaperones. Proc Natl Acad Sci USA 96, 7184-7189 (1999).

29. Blum, P., Ory, J., Bauemfeind, J. & Krska, J. Physiological consequences of DnaK and DnaJ overproduction in Escherichia coli. J Bacteriol 174, 7436-7444 (1992).

30. Grandvalet, C., Servant, P. & Mazodier, P. Disruption of hspR, the repressor gene of the dnaK operon in Streptomyces albus G. Mol Microbiol 23, 77-84 (1997).

31. VanBogelen, R. A., Acton, M. A. & Neidhardt, F. C. Induction of the heat shock regulon does not produce thermotolerance in Escherichia coli. Genes Dev. 1, 525-531 (1987).

32. Camacho, L. R., Ensergueix, D., Perez, E., Gicquel, B. & Guilhot, C. Identification of a virulence gene cluster of Mycobacterium tuberculosis by signature-tagged transposon mutagenesis. Mol Microbiol 34, 257-267 (1999).

33. Cox, J. S., Chen, B., McNeil, M. & Jacobs, W. R., Jr. Complex lipid determines tissue-specific replication of Mycobacterium tuberculosis in mice. Nature 402, 79-83 (1999).

34. Manabe, Y. C., Saviola, B. J., Sun, L., Murphy, J. R. & Bishai, W. R. Attenuation of virulence in Mycobacterium tuberculosis expressing a constitutively active iron repressor. Proc NatlAcadSci USA 96, 12844-12848 (1999).

35. Glickman, M. S., Cox, J. S. & Jacobs, W. R. A novel mycolic acid cyclopropane synthetase is required for cording, persistence, and virulence of Mycobacterium tuberculosis. Molecular Cell 5, 717-727 (2000).

36. McKinney, J. D. et al. Persistence of Mycobacterium tuberculosis in macrophages and mice requires the glyoxylate shunt enzyme isocitrate lyase. Nature 406, 735-738.(2000).

37. Parrish, N. M., Dick, J. D. & Bishai, W. R. Mechanisms of latency in Mycobacterium tuberculosis. Trends Microbiol 6, 107-112 (1998).

38 Wu, S. C., Ye, R., Wu, X. C., Ng, S. C. & Wong, S. L. Enhanced secretory production of a single-chain antibody fragment from Bacillus subtilis by coproduction of molecular chaperones. J Bacteriol 180, 2830-2835 (1998).

39. Baldwin, S. L. et al. Evaluation of new vaccines in the mouse and guinea pig model of tuberculosis. Infect Immun 66, 2951-2959 (1998).

40. Huang, Q., Richmond, J. F. L., Suzue, K., Eisen, H. N. & Young, R. A. In vivo cytotoxic T lymphocyte elicitation by mycobacterial heat shock protein 70 fusion proteins maps to a discrete domain and is CD4⁺ T cell independent. J Exp Med 191, 403-408 (2000).

41. Dye, C., Scheele, S., Dolin, P., Pathania, V. & Raviglione, M. C. Consensus statement. Global burden of tuberculosis: estimated incidence, prevalence, and mortality by country. WHO Global Surveillance and Monitoring Project. Jama 282, 677-686 (1999).

42. Mehlert, A. & Young, D. B. Biochemical and antigenic characterization of the Mycobacterium tuberculosis 71 kD antigen, a member of the 70 kD heat-shock protein family. Mol Microbiol 3, 125-130 (1989).

43. Dussurget, O. et al. Role of Mycobacterium tuberculosis copper-zinc superoxide dismutase. Infect Immun 69, 529-533 (2001).

44. De Smet, K. A., Kempsell, K. E., Gallagher, A., Duncan, K. & Young, D. B. Alteration of a single amino acid residue reverses fosfomycin resistance of recombinant MurA from Mycobacterium tuberculosis. Microbiology 145, 3177-3184 (1999).

45. Simmons, C. P. et al. Mucosal delivery of a respiratory syncytial virus CTL peptide with enterotoxin-based adjuvants elicits protective immunopathogenic, and immunoregulatory antiviral CD8⁺ T cell responses. J Immunol 166, 1106-1122 (2001).

46. Asea, A., Kraeft, S. K., Kurt-Jones, E. A., Stevenson, M. A., Chen, L. B., Finberg, R. W., Koo, G. C., and Calderwood, S. K. (2000) HSP70 stimulates cytokine production through a CD14-dependant pathway, demonstrating its dual role as a chaperone and cytokine. Nat Med 6: 435-442.

47. Bucca, G., Brassington, A. M., Schonfeld, H. J., and Smith, C. P. (2000) The HspR regulon of streptomyces coelicolor: a role for the DnaK chaperone as a transcriptional corepressordagger. Mol Microbiol 38: 1093-1103.

48. Castellino, F., Boucher, P. E., Eichelberg, K., Mayhew, M., Rothman, J. E., Houghton, A. N., and Germain, R. N. (2000) Receptor-mediated uptake of Antigen/Heat shock protein complexes results in major histocompatibility complex class I antigen presentation via two distinct processing pathways [In Process Citation]. J Exp Med 191: 1957-1964.

49. Chang, Z., Primm, T. P., Jakana, J., Lee, I. H., Serysheva, I., Chiu, W., Gilbert, H. F., and Quiocho, F. A. (1996) Mycobacterium tuberculosis 16-kDa antigen (Hsp16.3) functions as an oligomeric structure in vitro to suppress thermal aggregation. J Biol Chem 271: 7218-7223.

50. Cole, S. T., Brosch, R., Parkhill, J., Garnier, T., Churcher, C., Harris, D., Gordon, S. V., Eiglmeier, K., Gas, S., Barry, C. E., 3rd, Tekaia, F., Badcock, K., Basham, D., Brown, D., Chillingworth, T., Connor, R., Davies, R., Devlin, K., Feltwell, T., Gentles, S., Hamlin, N., Holroyd, S., Hornsby, T., Jagels, K., Barrell, B. G., and et al. (1998) Deciphering the biology of Mycobacterium tuberculosis from the complete genome sequence [see comments] [published erratum appears in Nature 1998 Nov. 12;396(6707):190]. Nature 393: 537-544.

51. Cunningham, A. F., and Spreadbury, C. L. (1998) Mycobacterial stationary phase induced by low oxygen tension: cell wall thickening and localization of the 16-kilodalton alphacrystallin homolog. J Bacteriol 180: 801-808.

52. Fernandes, N. D., Wu, Q. L., Kong, D., Puyang, X., Garg, S., and Husson, R. N. (1999) A mycobacterial extracytoplasmic sigma factor involved in survival following heat shock and oxidative stress. J Bacteriol 181: 4266-4274.

53. Grandvalet, C., de Crecy-Lagard, V., and Mazodier, P. (1999) The ClpB ATPase of Streptomyces albus G belongs to the HspR heat shock regulon. Mol Microbiol 31:521-532.

54. Grossman, A. D., Erickson, J. W., and Gross, C. A. (1984) The htpR gene product of E. coli is a sigma factor for heat-shock promoters. Cell 38: 383-390.

55. Hartl, F. U. (1996) Molecular chaperones in cellular protein folding. Nature 381: 571-579.

56. Hecker, M., Schumann, W., and Volker, U. (1996) Heat-shock and general stress response in Bacillus subtilis. Mol Microbiol 19: 417-428.

57. Helmann, J. D. (1999) Anti-sigma factors. Curr Opin Microbiol 2: 135-141.

58. Hinds, J., Mahenthiralingam, E., Kempsell, K. E., Duncan, K., Stokes, R. W., Parish, T., and Stoker, N. G. (1999) Enhanced gene replacement in mycobacteria. Microbiology 145:519-527.

59. Lee, B. Y., and Horwitz, M. A. (1995) Identification of macrophage and stress-induced proteins of Mycobacterium tuberculosis. J Clin Invest 96: 245-249.

60. Manganelli, R., Dubnau, E., Tyagi, S., Kramer, F. R., and Smith, I. (1999) Differentialexpression of 10 sigma factor genes in Mycobacterium tuberculosis. Mol Microbiol 31: 715-724.

61. Manganelli, R., Voskuil, M. I., Schoolnik, G. K., and Smith, I. (2001) The Mycobacterium tuberculosis ECF sigma factor sigmaE: role in global gene expression and survival in macrophages. Mol Microbiol 41: 423-437.

62. Monahan, I., Betts, J., Banerjee, D., and Butcher, P. (2001) Differential expression of mycobacterial proteins following phagocytosis by macrophages. Microbiology 147:459-471.

63. Narberhaus, F. (1999) Negative regulation of bacterial heat shock genes. Mol Microbiol 31:1-8.

64. Parish, T., and Stoker, N. G. (1997) Development and use of a conditional antisense mutagenesis system in mycobacteria. FEMS Microbiol Lett 154: 151-157.

65. Patel, B. K., Banerjee, D. K., and Butcher, P. D. (1991) Characterization of the heat shock response in Mycobacterium bovis BCG. J Bacteriol 173: 7982-7987.

66. Raman, S., Song, T., Puyang, X., Bardarov, S., Jacobs, W. R., Jr., and Husson, R. N. (2001) The alternative sigma factor SigH regulates major components of oxidative and heat stress responses in Mycobacterium tuberculosis. J Bacteriol 183: 6119-6125.

67. Sherman, D. R., Voskuil, M., Schnappinger, D., Liao, R., Harrell, M. I., and Schoolnik, G. K. (2001) Regulation of the Mycobacterium tuberculosis hypoxic response gene encoding alpha-crystallin. Proc Natl Acad Sci USA 98: 7534-7539.

68. Stewart, G. R., Snewin, V. A., Walzl, G., Hussell, T., Tormay, P., O'Gaora, P., Goyal, M., Betts, J., Brown, I. N., and Young, D. B. (2001) Overexpression of heat-shock proteins reduces survival of Mycobacterium tuberculosis in the chronic phase of infection. Nature Medicine 7: 732-737.

69. Stover, C. K., de la Cruz, V. F., Bansal, G. P., Hanson, M. S., Fuerst, T. R., Jacobs, W. R., Jr., and Bloom, B. R. (1992) Use of recombinant BCG as a vaccine delivery vehicle. Adv Exp Med Biol 327: 175-182.

70. Sudre, P., ten Dam, G., and Kochi, A. (1992) Tuberculosis: a global overview of the situation today. Bull World Health Organ 70: 149-159.

71. Suto, R., and Srivastava, P. K. (1995) A mechanism for the specific immunogenicity of heat shock protein-chaperoned peptides. Science 269: 1585-1588.

72. Wards, B. J., and Collins, D. M. (1996) Electroporation at elevated temperatures substantially improves transformation efficiency of slow-growing mycobacteria. FEMS Microbiol Lett 145: 101-105.

73. Wilson, M., DeRisi, J., Kristensen, H. H., Imboden, P., Rane, S., Brown, P. O., and Schoolnik, G. K. (1999) Exploring drug-induced alterations in gene expression in Mycobacterium tuberculosis by microarray hybridization. Proc Natl Acad Sci USA 96: 12833-12838.

74. Wilson M., Voskuil M., Schnappinger D., Schoolnik G K (2001) Functional genomics of Mycobacterium tuberculosis using DNA microarrays in: Methods in Molecular Medicine, vol 54: Mycobacterium tuberculosis Protocols (eds: T. Parish & N. G. Stoker) Humana Press Inc, Totowa, N.J. pp335-357.

75. Yang, H., Huang, S., Dai, H., Gong, Y., Zheng, C., and Chang, Z. (1999) The Mycobacterium tuberculosis small heat shock protein Hsp16.3 exposes hydrophobic surfaces at mild conditions: conformational flexibility and molecular chaperone activity. Protein Sci 8: 174-179.

76. Young, D. B., and Garbe, T. R. (1991) Heat shock proteins and antigens of Mycobacterium tuberculosis. Infect Immun 59: 3086-3093.

77. Yuan, Y., Crane, D. D., and Barry, C. E., 3rd (1996) Stationary phase-associated protein expression in Mycobacterium tuberculosis: function of the mycobacterial alphacrystallin homolog. J Bacteriol 178: 4484-4492.

78. Yuan, Y., Crane, D. D., Simpson, R. M., Zhu, Y. Q., Hickey, M. J., Sherman, D. R., and Barry, C. E., 3rd (1998) The 16-kDa alpha-crystallin (Acr) protein of Mycobacterium tuberculosis is required for growth in macrophages. Proc Natl Acad Sci USA 95:9578-9583.

We claim:

1. An immunogenic composition comprising mycobacteria wherein said mycobacteria comprises modified protein production.

2. The composition of claim 1, wherein the modified protein expression comprises an increase in heat shock protein production.

3. The composition of claim 2, wherein the heat shock protein is selected from the group consisting of Hsp10, Hsp40, Hsp60, Hsp70, Hsp90, GrpE, ClpB and alpha-cystallin.

4. The composition of claim 1, wherein the mycobacteria is selected from the group consisting of M. tuberculosis, M. avium-intracellulare, M. bovis, M. kansasii, M. fortuitum, M. chelonae, M. leprae, M. africanum, M. microti and M. paratuberculosis.

5. The composition of claim 1, wherein the mycobacteria comprises M. tuberculosis.

6. The composition of claim 5, wherein the heat shock protein comprises Hsp 60 or Hsp 70.

7. The composition of claim 5, wherein the heat shock protein consists of Hsp 60 and Hsp 70.

8. The composition of claim 1, further comprising a pharmaceutically acceptable carrier.

9. A method for eliciting an immune response in a human or animal comprising to said human or animal an immunogenic composition wherein said composition comprises an pathogenic organism having modified heat shock protein production.

10. The method of claim 9, wherein the pathogenic organism is selected from the group consisting of M. tuberculosis, M. avium-intracellulare, M. bovis, M. kansasii, M. fortuitum, M. chelonae, M. leprae, M. africanum, M. microti and M. paratuberculosis.

11. The method of claim 10, wherein the pathogenic organism comprises M. tuberculosis and the modified heat shock protein production comprises an increase in the production of heat shock proteins.

12. The method of claim 11, wherein the heat shock protein is selected from the group consisting of Hsp10, Hsp40, Hsp60, Hsp70, Hsp90, GrpE, ClpB and alpha-cystallin.

13. The method of claim 11, wherein the heat shock proteins consists of Hsp 60 and Hsp 70.

14. A method for treating mycobacterial disease comprising administering to a human or animal an immunogenic composition comprising modified mycobacterial pathogens wherein said mycobacterial pathogens have increased heat shock protein production.

15. The method of claim 14, wherein the mycobacterial disease is selected from the group consisting of tuberculosis and Crohn's disease.

16. The method of claim 15, wherein the heat shock protein is selected from the group consisting of Hsp10, Hsp40, Hsp60, Hsp70, Hsp90, GrpE, ClpB and alpha-cystallin.

17. The method of claim 15, wherein the heat shock protein consists of Hsp 60 and Hsp 70.

18. The method of claim 14, further comprising a pharmaceutically acceptable carrier.

19. An immunogenic composition comprising an improved BCG vaccine wherein the vaccine comprises modified M. bovis having increased heat shock protein production.

20. The immunogenic composition of claim 19, wherein the heat shock protein is selected from the group consisting of Hsp10, Hsp40, Hsp60, Hsp70, Hsp90, GrpE, ClpB and alpha-cystallin.
